SEQUENCE LISTING (36 sequences)

SEQ ID NO: 1 - DNA, 21 bp, Artificial sequence
  synthetic sequence
ttgctgttgaagtctcaggag

SEQ ID NO: 2 - DNA, 19 bp, Artificial sequence
  synthetic sequence
tgccgctgatgggtttgac

SEQ ID NO: 3 - DNA, 21 bp, Artificial sequence
  synthetic sequence
ttgctcggcatattctgagcc

SEQ ID NO: 4 - DNA, 22 bp, Artificial sequence
  synthetic sequence
tggcactggtatgaaatcagac

SEQ ID NO: 5 - DNA, 19 bp, Artificial sequence
  synthetic sequence
cctcggcccaagatcctac

SEQ ID NO: 6 - DNA, 20 bp, Artificial sequence
  synthetic sequence
gggacccgctgtcttctagt

SEQ ID NO: 7 - DNA, 21 bp, Artificial sequence
  synthetic sequence
attcctggtaaccgaatgctg

SEQ ID NO: 8 - DNA, 20 bp, Artificial sequence
  synthetic sequence
ggtctcaacccccagctagt

SEQ ID NO: 9 - DNA, 23 bp, Artificial sequence
  synthetic sequence
cttactgactggcatgaggatca

SEQ ID NO: 10 - DNA, 21 bp, Artificial sequence
  synthetic sequence
tcagcgtgtccaaacactgag

SEQ ID NO: 11 - DNA, 19 bp, Artificial sequence
  synthetic sequence
gccacggcacagtcattga

SEQ ID NO: 12 - DNA, 21 bp, Artificial sequence
  synthetic sequence
ggtagctctctgcctgatgag

SEQ ID NO: 13 - DNA, 19 bp, Artificial sequence
  synthetic sequence
tcccgctttctggtgacag

SEQ ID NO: 14 - DNA, 21 bp, Artificial sequence
  synthetic sequence
gctccaaaggacttgtacgtg

SEQ ID NO: 15 - DNA, 20 bp, Artificial sequence
  synthetic sequence
cagggcctttaccagggaga

SEQ ID NO: 16 - DNA, 21 bp, Artificial sequence
  synthetic sequence
aaattggaggtgttcgtccca

SEQ ID NO: 17 - DNA, 20 bp, Artificial sequence
  synthetic sequence
aagccacctttcggatgagg

SEQ ID NO: 18 - DNA, 20 bp, Artificial sequence
  synthetic sequence
ccaggagacaccaggatgaa

SEQ ID NO: 19 - DNA, 20 bp, Artificial sequence
  synthetic sequence
tgtgtccgtcgtggatctga

SEQ ID NO: 20 - DNA, 21 bp, Artificial sequence
  synthetic sequence
gctgtggtagttacaggggtc

SEQ ID NO: 21 - DNA, 23 bp, Artificial sequence
  synthetic sequence
tggcgtcgatcatctgattctaa

SEQ ID NO: 22 - DNA, 23 bp, Artificial sequence
  synthetic sequence
caaggtatcctctggtgctaaag

SEQ ID NO: 23 - DNA, 21 bp, Artificial sequence
  synthetic sequence
cctagacatccagaggtgaca

SEQ ID NO: 24 - DNA, 22 bp, Artificial sequence
  synthetic sequence
tcaactcaaattcgctgaggac

SEQ ID NO: 25 - DNA, 23 bp, Artificial sequence
  synthetic sequence
ccggtctcaggtatcaaactagc

SEQ ID NO: 26 - DNA, 23 bp, Artificial sequence
  synthetic sequence
gccgatgatctctctcaagtgat

SEQ ID NO: 27 - DNA, 20 bp, Artificial sequence
  synthetic sequence
gcagctctaggagcatgtgg

SEQ ID NO: 28 - DNA, 21 bp, Artificial sequence
  synthetic sequence
cgccaagggagttaaagactt

SEQ ID NO: 29 - DNA, 21 bp, Artificial sequence
  synthetic sequence
tgctgatggcctgattgtctt

SEQ ID NO: 30 - DNA, 23 bp, Artificial sequence
  synthetic sequence
tggtaggaactgatggggatatt

SEQ ID NO: 31 - DNA, 21 bp, Artificial sequence
  synthetic sequence
acctgagcctttacaaggacc

SEQ ID NO: 32 - DNA, 21 bp, Artificial sequence
  synthetic sequence
tgatctgaagggcagcatttc

SEQ ID NO: 33 - DNA, 21 bp, Artificial sequence
  synthetic sequence
tgtgtgctggatgtgatgttc

SEQ ID NO: 34 - DNA, 19 bp, Artificial sequence
  synthetic sequence
tccccgtctgagcctaagg

SEQ ID NO: 35 - DNA, 19 bp, Artificial sequence
  synthetic sequence
cgcagcagtcgttgttctg

SEQ ID NO: 36 - DNA, 20 bp, Artificial sequence
  synthetic sequence
tcatgtcagtgtaggctcgc

(19) United States
(12) Patent Application Publication
(10) Pub. No.: US 2022/0267428 A1
(43) Pub. Date: Aug. 25, 2022

(54) BLOCKADE OF RGMb FOR TREATING INFLAMMATORY BOWEL DISEASE AND COLITIS

(71) Applicant: The Board of Trustees of the Leland Stanford Junior University, Stanford, CA (US)

(72) Inventors: Everett Hurteau Meyer, Belmont, CA (US); Magdiel Perez Cruz, Stanford, CA (US)

(21) Appl. No.: 17/281,464

(22) PCT Filed: Oct. 10, 2019
(86) PCT No.: PCT/US2019/055686

§ 371 Date: Mar. 30, 2021

Related U.S. Application Data
(60) Provisional application No. 62/743,867, filed on Oct. 10, 2018.

(51) Int. Cl. C07K 16/22; A61P 1/00

(52) U.S. Cl. CPC C07K 16/22; A61P 1/00; A61K 2039/505

(57) ABSTRACT

RGMb antagonists reduce undesirable immune responses associated with autoimmune conditions, including inflammatory bowel diseases such as colitis.

### CROSS REFERENCE

This application is a 371 Application and claims the benefit of PCT Application No. PCT/US2019/055686, filed Oct. 10, 2019, which claims the benefit of U.S. Provisional Application No.62/743,867, filed Oct. 10, 2018, which is-are incorporated herein by reference in its their entirety.

### INCORPORATION BY REFERENCE OF SEQUENCE LISTING PROVIDED AS A TEXT FILE

A Sequence Listing is provided herewith in a text file, (S18-408_STAN-1508_SEQ_LIST_ST25) created on (Nov. 9, 2021) and having a size of (8,000 bytes of file). The contents of the text file are incorporated herein by reference in its entirety.

### BACKGROUND

Therapeutics to interrupt major signaling pathways involved in T cell maturation, proliferation and polarization are the backbone of modern strategies to prevent transplantation rejection. Many of the major signaling pathways have been identified including the CD28:B7, CTLA-4:B7, ICOS:B7h, PD-1:PDL, CD134:CD134L, 4-1BB:4-1BBL, CD27:CD70 pathway. In contrast to these pathways where the mechanism of action is well described, a number of studies implicate the bone morphogenetic proteins (BMPs) and neogenin pathways as strong drivers of innate and adaptive inflammation but these are observational studies and it is unclear if these pathways could be involved in transplantation tolerance. Recently, BMP2, BMP4, neogenin and the immune costimulatory molecule PDL-2 have been found to bind to the repulsive guidance molecule b (RGMb) signaling hub and regulate pulmonary mucosal immunity.

BMPs have diverse roles in many physiologic and pathologic processes, including cell proliferation, differentiation and apoptosis. RGMb is a member of the RGM family, which consists of RGMa, RGMb (Dragon), and RGMc (hemojuvelin). RGMb has a well elucidated role in neural development and due to this RGMb knock-out mice die 2-3 weeks after birth. In the adult mice, RGMb neural expression is quite limited to the basal ganglia and pituitary, but the molecule is expressed more broadly in the gut, bone, heart, lung, liver, kidney, testis, ovary, uterus, epididymis, and pancreas of adult mice and human. Recent studies have shown that RGMb is expressed by immune cell subsets including macrophages.

All three RGM members function as coreceptors that enhance BMP signaling, which is generally proliferative. The crystal structure and binding regions of RGMb were recently reported and reveal that a complex signaling hub. In one extracellular region of the molecule, RGMb binds to BMP2, BMP4 and neogenin in a complex and this interaction can be blocked by a monoclonal antibody 9D1. At a different extracellular region, RGMb binds to PDL-2 and this interaction can be blocked by a different monoclonal antibody 2C9. Intracellularly, RGMb also complexes with BMP type II and type I receptors and ActRIIA increasing BMP signaling.

Inflammatory bowel disease (IBD) is an increasingly common inflammatory disorder of the gastrointestinal tract, with symptoms including weight loss, watery diarrhea, rectal bleeding, abdominal cramps, abdominal pain, and fever. IBD affects both children and adults, and according the Mayo Clinic, more than 1.5 million Americans have Crohn's disease or ulcerative colitis, the most common forms of inflammatory bowel disease. IBD may at times begin clinically with a more benign or milder presentation, resembling Irritable Bowel Syndrome (IBS) but can subsequently progress with increasing inflammation, which is distinct from IBS. The precise cause of IBD remains unknown. In some cases, IBD requires surgical intervention.

IBD is difficult to treat effectively, and treatment of IBD is varied. Treatment typically includes salicylate derivatives (e.g. 5-ASA) given orally or rectally, and/or corticosteroids, despite known problematic side-effects.

Chronic pancreatitis is commonly defined as a continuing, chronic, inflammatory process of the pancreas, characterized by irreversible morphologic changes. This chronic inflammation can lead to chronic abdominal pain and/or impairment of endocrine and exocrine function of the pancreas. Chronic pancreatitis usually is envisioned as an atrophic fibrotic gland with dilated ducts and calcifications. However, findings on conventional diagnostic studies may be normal in the early stages of chronic pancreatitis, as the inflammatory changes can be seen only by histologic examination. Based on estimates from hospital discharge data in the United States, approximately 87,000 cases of pancreatitis occur annually. Roughly half of the patients with chronic pancreatitis eventually require surgical intervention, which is indicated when an anatomical complication (e.g., pancreatic pseudocyst, abscess, fistula, ascites, fixed obstruction of the intrapancreatic portion of the distal common bile duct, stenosis of the duodenum with gastric outlet obstruction) that is correctable by a mechanical intervention exists.

There remains an unmet need in the art for improved methods for treating Inflammatory gastrointestinal diseases. This is addressed herein.

### SUMMARY

Compositions and methods are provided for reducing undesirable immune responses associated with inflammatory gastrointestinal diseases, including inflammatory bowel diseases (IBD), e.g. Crohn's disease, ulcerative colitis, etc., chronic pancreatitis, and the like. In the methods of the invention, an antagonist of RGMb is administered to an individual in a dose effective to reduce undesirable immune responses associated with inflammatory diseases, which immune responses can initiate and/or maintain the inflammatory disease. It is shown herein that blockade of RGMb with an antagonistic antibody protects against development of such inflammatory disease, and reduces undesirable inflammation during the course of the disease.

In some embodiments methods are provided comprising administering an effective dose of an antagonist of RGMb to an individual suffering from an inflammatory gastrointestinal disease; or to an individual at risk of developing an inflammatory gastrointestinal disease, where the dose and dosage regimen is effective to reduce inflammation and/or symptoms of disease in the recipient. The symptoms relieved by the provided methods include weight loss, colon shortening, soft/loose stool (e.g., diarrhea, watery diarrhea, etc.), rectal bleeding (e.g., bloody stool), abdominal cramps, abdominal pain, vomiting, acute right lower quadrant pain, malaise, fatigue, fever, and/or anemia.

The present invention relates to blockade of Repulsive Guidance Molecule (RGMb, Dragon), which is one of the three repulsive guidance molecule (RGM) family members, and is a glycophosphatidylinositol-anchored membrane proteins. RGMb is a bone morphogenetic protein (BMP) coreceptor and sensitizer of BMP signaling. In some embodiments, blockade of RGMb is achieved by contacting cells with an antagonist of RGMb. In some embodiments an antagonist is a polypeptide, including without limitation an antibody, a competitive inhibitor of BMP-2/4 binding, etc. In some embodiments an antibody is human, humanized, or chimeric. An antibody comprising a human Fc region is of interest for treatment of a human patient. Antibodies may be selected for low activation through the Fc receptor; or alternatively may be selected to be active in CDC, ADCC, ADCP, etc. In some embodiments an antagonistic antibody interferes with signaling mediated by RGMb, e.g. by blocking binding of RGMb to BMP-2/4. An antibody may bind to the BMP2,4 binding site on RGMb. In other embodiments blockade is achieved with a polynucleotide, e.g. an anti-sense oligonucleotide, an RNAi, and the like.

In some embodiments, the invention provides an RGMb antagonist, including without limitation an antibody that specifically binds to RGMb and blocks activity, as well as pharmaceutical formulations of the same. In another aspect, the invention provides pharmaceutical formulations containing one or more RGMb antagonist(s) and a pharmaceutically acceptable carrier, which may be provided in a unit dose formulation suitable for treatment of inflammatory gastrointestinal disease. The formulation may comprise one or more active agents or a mixture or “cocktail” of agents having different activities, e.g. including one or more additional therapeutic agents. The formulation may be provided in a unit dose, e.g. an effective dose of an agonist in a sterile container suitable for clinical use, and may be included in a kit format.

In some embodiments, an RGMb antagonist is administered in combination with at least one other anti-IBD or gastrointestinal agent. Suitable anti-IBD agents for combination therapy include: 5-aminosalicylic acid (5-ASA); 5-ASA derivatives (e.g., sulfasalazine, mesalamine, balsalazide, olsalazine); antibiotics (e.g., metronidazole, ciprofloxacin, rifaximin); corticosteroids (e.g., hydrocortisone, prednisone, methylprednisolone, prednisolone, entocort (budesonide), dexamethasone); immunosuppressants (e.g., azathioprine, 6-mercaptopurine, methotrexate, cyclosporine); DMARDs, TNF Inhibitors (e.g., adalimumumab, certolizumab pegol, golimumab, infliximab, and infliximab-dyyb); anti-integrins including natalizumab and vedolizumab; histamine h2 antagonists (e.g., cimetidine, ranitidine, famotidine, nizatidine); proton pump inhibitors (e.g., omeprazole, lansoprazole, esomeprazole magnesium, rabeprazole sodium, pantoprazole); antidiarrheals (e.g., diphenoxylate and atropine, loperamide, cholestyramine); anticholinergic, antispasmodic agents (e.g., dicyclomine, hyoscyamine); and the like.

These and other aspects and embodiments of the invention are described in more detail below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-FIG. 1B. Total body irradiation overexpression of RGMb in gut. A) To analysis the TBI effect in RGMb expression in spleen and gut, BMPR1a and RGMb RNA expression was quantified by qPCR at 6 and 24 h post-TBI and B) RGMb protein expression in gut at 24 and 48 h post-TBI by western blotting. Results are expressed as mean±SEM (fold to control) (n=3). *p<0.05, **p<0.01.

FIG. 2A-FIG. 2F. Treatment with anti-RGMb-BMP2,4 binding site antibody induces tolerance in GvHD. A) To assess the capacity of anti-RGMb-BMP2,4 or anti-RGMb-PL2 binding site antibody treatment to prevent GvHD, mice were injected with 9D1 or 2C9 antibody (400 μg/mouse; i.p.). B) Mouse survival was monitored during the 60 days post-BM transplantation also C) the body weight. D) Gut histopathology was performed by H&E staining on control isotype antibody-, 9D1- and 2C9-treated mice at 9 days post-BM transplantation. Histology is shown at 200× and E) Histological score. F) To assess the impact of anti-RGMb-BMP2,4 binding site antibody treatment in tolerance induction, female NSG mice were injected with anti-RGMb antibody (400 μg/mouse; i.p.) and 24 h before the human PBMC transplantation. They received PBMC (5×10⁶ cells; i.v.) and were sacrificed at day 34. Mortality was monitored during the 34 days post-transplantation. Results are expressed as mean±SEM (n≥3). *p<0.05, **p<0.01 compared to control isotype treated-mice.

FIG. 3A-FIG. 3D. Anti-RGMb-BMP2,4 binding site antibody therapy protects against leukemia. A) To assess the protection of anti-RGMb-BMP2,4 binding site antibody treatment against tumor, mice were injected with 9D1 antibody (400 μg/mouse; i.p.) and after the first injection they received luc⁺ A20 cells B) Mice survival was monitored during the 31 days post-transplantation. C) Representative image and D) Quantification of signal was done using living imaging program. Results are expressed as mean±SEM (n≥3). *p<0.05, **p<0.01 compared to control isotype treated-mice.

FIG. 4A-FIG. 4F. BMP signaling pathway blockage reduces T cell proliferation. To assess the impact of anti-RGMb antibody treatment in T cell proliferation, CD11 b⁺ cells were isolated from BALB/c mice and were incubated with naïve T cells from C57BI/6 mice. Mixed cells were cultured for 7 days with or without anti-RGMb antibody (100 μg/ml). A) T cells proliferation. Also mice were injected with anti-RGMb-BMP2,4 or anti-RGMb-PDL2 binding site antibody (400 μg/mouse; i.p.) and after the first infection they received bone marrow and luc⁺ T cells by i.v. route. B) Representative bioluminescence images of luc⁺ T conventional cells biodistribution analyzed by Xenogen IVIS 100 in vivo imaging system and quantification of signal was done using living imaging program. D) percentage of CD3⁺ T cells in gut and E) percentage of CD4⁺ and CD8⁺ T cells in gut were analyzed by cytometry. F) Heat map generated from DNA microarray data reflecting gene expression values in gut after 9 days of BM transplantation. Data are expressed as bi-weight avg signal (log 2). *p<0.05, **p<0.01 compared to control isotype treated-mice.

FIG. 5A-FIG. 5E. Treatment with anti-RGMb-BMP2,4 binding site antibody prevents inflammatory bowel diseases. A) To assess the capacity of anti-RGMb antibody treatment to prevent colitis, mice were injected with anti-BMP2,4 binding site or isotype control antibody (200 μg/mouse; i.p.). 24 h after first injection mice were treated or not with 2.5% DSS in their drinking water for 7 days to induce colitis. Mice were euthanized at day 8. B) Survival was monitored during the 21 days post-DSS administration. C) body weight changes during induction of colitis. Statistic of the weight changes on day 8 was determined using the student's test. D) Gross morphological changes of colon on day 8 after 7 days of 2.5% DSS and 1 day drinking water. E) Gut histopathology was performed by H&E and RGMb staining on control isotype antibody- and 9D1-treated mice at 9 days post-DSS administration. Histology is shown at 200×. Results are expressed as mean±SEM (n≥3). *p<0.05, **p<0.01 compared to control isotype treated-mice.

FIG. 6A-FIG. 6C. Treatment with anti-RGMb-BMP2,4 binding site antibody promote anti-inflammatory response in DSS-induced colitis. A) Frequency of naïve and effector memory CD4⁺ cells in spleen and B) colon. C). Gene expression was validated by qPCR in colon after 7 days of DSS-induced colitis. Data are expressed as relative expression to GAPH. Data are expressed as mean±SEM (n≥4). *p<0.05, **p<0.01 compared to control isotype treated-mice.

FIG. 7A-FIG. 7D. Anti-RGMb antibody therapy delays T cell maturation. To assess the impact of anti-RGMb antibody treatment in T cell maturation, CD11b⁺ cells were isolated from BALB/c mice and were incubated with naïve T cells from C57BI/6 mice. Mixed cells were cultured for 7 days with or without anti-RGMb antibody (100 μg/ml). A) Maturation of T cells, B) Memory and C) Naïve T cells but also E) cytokine profile were monitored after the 7 days of mixed lymphocyte reaction. D) Representative Dot plot of cytometry after 7 days of treatment. *p<0.05, **p<0.01 compared to control isotype treated-mice.

FIG. 8. List of genes printed on the microarray. Fold change was calculated by RGMb versus Tcon Bi-weight Avg Signal (Log 2).

FIG. 9. Primers for Real-time PCR.

FIG. 10. Treatment with anti-RGMb-BMP2,4 binding site antibody reduce CD8⁺ T cell population in spleen in GvHD model. Results are expressed as mean±SEM (n≥3).

FIG. 11. Treatment with anti-RGMb-BMP2,4 binding site antibody promote anti-inflammatory response in GvHD model. Results are expressed as mean±SEM (n≥3). **p<0.01 compared to control isotype treated-mice.

FIG. 12. Treatment with anti-RGMb-BMP2,4 binding site antibody promote anti-inflammatory response in inflammatory bowel diseases model. Results are expressed as mean±SEM (n≥3). *p<0.05, **p<0.01 compared to control isotype treated-mice.

FIG. 13. Treatment with anti-RGMb-BMP2,4 binding site antibody reduce cytokine production after a mixed lymphocyte reaction between CD11b⁺ cells and naïve T cells. Results are expressed as mean±SEM (n=3). *p<0.05, **p<0.01 compared to control isotype treated-mice.

### DETAILED DESCRIPTION OF THE EMBODIMENTS

### Definitions

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

As used herein, the term “prevention” refers to alleviating the disease or condition from occurring in a subject which has not yet been diagnosed as having it. As used herein, the term “subject” denotes a mammal, such as a rodent, a feline, a canine, and a primate. Preferably a subject according to the invention is a human.

The terms “individual,” “subject,” “host,” and “patient,” are used interchangeably herein and refer to any mammalian subject for whom diagnosis, treatment, or therapy is desired, particularly humans.

An “effective amount” is an amount sufficient to effect beneficial or desired clinical results. An effective amount can be administered in one or more administrations, and may be administered for a period of time sufficient to effect a therapeutic results, e.g. for up to about one week, up to about 2 weeks, up to about 3 weeks, or more. For purposes of this invention, an effective amount is an amount, delivered in an effective regimen, that is sufficient to palliate, ameliorate, stabilize, reverse, prevent, slow or delay the progression of the disease state (e.g., IBD and/or chronic pancreatitis) by decreasing IBD-associated clinical symptoms (e.g., weight loss, colon shortening, soft/loose stool (e.g., diarrhea, watery diarrhea, etc.), rectal bleeding (e.g., bloody stool), abdominal cramps, abdominal pain, vomiting, acute right lower quadrant pain, malaise, fatigue, fever, and/or anemia) and/or clinical symptoms associated with chronic pancreatitis (e.g., chronic abdominal pain and/or symptoms associated with impairment of endocrine and exocrine function of the pancreas).

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals with autoimmune diseases, including IBD or chronic pancreatitis, and the like. Subjects may also include other mammals, particularly those mammals useful as laboratory models for human disease, e.g. mouse, rat, cynomolgus monkey, dog, etc.

“In combination with”, “combination therapy” and “combination products” refer, in certain embodiments, to the concurrent administration to a patient of the agents described herein. When administered in combination, each component can be administered at the same time or sequentially in any order at different points in time. Thus, each component can be administered separately but sufficiently closely in time so as to provide the desired therapeutic effect.

“Concomitant administration” of active agents in the methods of the invention means administration with the reagents at such time that the agents will have a therapeutic effect at the same time. Such concomitant administration may involve concurrent (i.e. at the same time), prior, or subsequent administration of the agents. A person of ordinary skill in the art would have no difficulty determining the appropriate timing, sequence and dosages of administration for particular drugs and compositions of the present invention.

Inflammatory Bowel Disease. As used herein, the term “inflammatory bowel disease” or “IBD” refers to any of a variety of diseases characterized by inflammation of all or part of the intestines (e.g., colon and/or small intestine). Non-limiting examples of “IBD” include: Crohn's Disease, Ulcerative Colitis, radiation colitis, Collagenous colitis, Lymphocytic colitis, Ischaemic colitis, Diversion colitis, Behçet's disease, and Indeterminate colitis. As will be understood by one of ordinary skill in the art, the two IBD types that account for the majority of IBD clinical cases are Crohn's Disease and Ulcerative Colitis. While IBD symptoms vary from patient to patient and some may be more common than others, the symptoms can include weight loss, colon thickening, soft/loose stool (e.g., diarrhea, watery diarrhea, etc.), rectal bleeding (e.g., bloody stool), abdominal cramps, abdominal pain, vomiting, acute right lower quadrant pain, malaise, fatigue, fever, and/or anemia.

Different forms of IBD differ in the location and nature of the inflammatory changes. For example, Crohn's disease can affect any part of the gastrointestinal tract, from mouth to anus, although a majority of the cases start in the terminal ileum. Crohn's disease can also affect the entire thickness of the bowel wall. In addition, in Crohn's disease, the inflammation of the intestine can “skip” leaving normal areas in between patches of diseased intestine (sometimes referred to as skip lesions). In more severe cases, Crohn's can lead to tears (fissures) in the lining of the anus, which may cause pain and bleeding, especially during bowel movements. Inflammation may also cause a fistula to develop. In contrast, ulcerative colitis is restricted to the colon and the rectum. Microscopically, ulcerative colitis is restricted to the mucosa (epithelial lining of the gut), while Crohn's disease can affect the whole bowel wall (“transmural lesions”). In patients with ulcerative colitis, the lining of the colon can become inflamed and develop tiny open sores, or ulcers, that produce pus and mucous. The combination of inflammation and ulceration can cause abdominal discomfort and frequent emptying of the colon. Crohn's disease and ulcerative colitis can present with extra-intestinal manifestations (e.g., liver problems, arthritis, skin manifestations, eye problems, etc.). Rarely, a definitive diagnosis of neither Crohn's disease nor ulcerative colitis can be made because of idiosyncrasies in the presentation. In this case, a diagnosis of indeterminate colitis may be made.

IBD is associated with inflammation of the gastrointestinal tract, and encompasses acute and chronic inflammatory conditions. Acute inflammation is generally characterized by a short time of onset and infiltration or influx of neutrophils. Chronic inflammation is generally characterized by a relatively longer period of onset and infiltration or influx of mononuclear cells. Chronic inflammation is also typically characterized by periods of spontaneous remission and spontaneous occurrence. “Mucosal layer of the gastrointestinal tract” is meant to include mucosa of the bowel (including the small intestine and large intestine), rectum, stomach (gastric) lining, oral cavity, and the like.

“Chronic IBD” refers to IBD that is characterized by a relatively longer period of onset, is long-lasting (e.g., from several days, weeks, months, or years and up to the life of the subject), and is associated with infiltration or influx of mononuclear cells and can be further associated with periods of spontaneous remission and spontaneous occurrence. Thus, subjects with chronic IBD may be expected to require a long period of supervision, observation, or care.

In some embodiments of the invention, an individual is diagnosed with a chronic inflammatory disease of the bowels prior to treatment. In some embodiments, a patient is diagnosed with ulcerative colitis. In other embodiments, a patient is diagnosed with Crohn's disease.

Diagnosis is suggested by typical symptoms and signs, particularly when accompanied by extraintestinal manifestations or a history of previous similar attacks. UC should be distinguished from Crohn disease but more importantly from other causes of acute colitis (eg, infection; in elderly patients, ischemia). In all patients, stool cultures for enteric pathogens should be done, and Entamoeba histolytica should be excluded by examination of fresh stool specimens. Sigmoidoscopy allows visual confirmation of colitis and permits direct sampling of stool or mucus for culture and microscopic evaluation, as well as biopsy of affected areas. Although visual inspection and biopsies may be nondiagnostic, because there is much overlap in appearance among different types of colitis, acute, self-limited, infectious colitis can usually be distinguished histologically from chronic idiopathic UC or Crohn colitis. Severe perianal disease, rectal sparing, absence of bleeding, and asymmetric or segmental involvement of the colon indicate Crohn disease rather than UC.

X-rays are not diagnostic but occasionally show abnormalities. Plain x-rays of the abdomen may show mucosal edema, loss of haustration, and absence of formed stool in the diseased bowel. Barium enema shows similar changes, albeit more clearly, and may also show ulcerations. A shortened, rigid colon with an atrophic or pseudopolypoid mucosa is often seen after several years of illness. X-ray findings of thumbprinting and segmental distribution are more suggestive of intestinal ischemia or possibly Crohn colitis rather than of UC.

Chronic Pancreatitis. The term “chronic pancreatitis” is used herein as commonly defined: a continuing, chronic, inflammatory process of the pancreas, characterized by irreversible morphologic changes. This chronic inflammation can lead to chronic abdominal pain and/or impairment of endocrine and exocrine function of the pancreas. Chronic pancreatitis is usually seen as an atrophic fibrotic gland with dilated ducts and calcifications. However, findings on conventional diagnostic studies may be normal in the early stages of chronic pancreatitis, as the inflammatory changes can be seen only by histologic examination. Based on estimates from hospital discharge data in the United States, approximately 87,000 cases of pancreatitis occur annually. Roughly half of the patients with chronic pancreatitis eventually require surgical intervention, which is indicated when an anatomical complication (e.g., pancreatic pseudocyst, abscess, fistula, ascites, fixed obstruction of the intrapancreatic portion of the distal common bile duct, stenosis of the duodenum with gastric outlet obstruction) that is correctable by a mechanical intervention exists.

RGMb. Repulsive guidance molecules (RGMs) compose a family of glycosylphosphatidylinositol (GPI)-anchored axon guidance molecules and perform several functions during neural development. RGMb (DRAGON) is a member of the family which is expressed early in the developing nervous system. Bone morphogenetic proteins (BMPs) are members of the transforming growth factor (TGF) beta superfamily of ligands that regulate many crucial aspects of embryonic development and organogenesis. RGMb binds directly to BMP2 and BMP4 but not to BMP7 or other TGFβ ligands. RGMb also associates directly with BMP type I (ALK2, ALK3, and ALK6) and type II (ActRII and ActRIIB) receptors, and its signaling is reduced by dominant negative Smad1 and ALK3 or -6 receptors. The direct interaction of RGMb with BMP ligands and receptors indicates that it is a BMP co-receptor that potentiates BMP signaling.

PD-L2 is another ligand of RGMb, but PD-L2 and BMP-2/4 bind to distinct sites on RGMb. Antibodies to mouse RGMb (see Xiao et al. (2014) JEM 211:943-959, herein specifically incorporated by reference) are known in the art to block the interaction of RGMb with its ligands. For example the antibody 9D1 and 8B2 block RGMb binding to BMP-2/4 in an ELISA and are dual blockers of RGMb interactions with PD-L2 and BMP-2/4. However, analysis has shown that PD-L2-Ig fusion protein or anti-PDL2 antibodies do not block RGMb binding to BMP-2/4, suggesting that the binding sites on RGMb for PD-L2 and BMP are close but distinct. Antibodies that are specific for PD-L2, which block the interaction of PD-L2 with RGMb, but do not affect the binding of BMP-2/4 with RGMb, include for example mAb 2C9.

The genetic sequence of human RGMb may be accessed at Genbank, NM_001012761, see Samad et al. (2004) J. Neurosci. 24 (8), 2027-2036 and Samad et al. (2005) J. Biol. Chem. 280 (14), 14122-14129, each herein specifically incorporated by reference.

By RGMb inhibitory agent or antagonist is meant an agent that inhibits the activity, e.g. binding to; interfering with binding partners; reducing expression; reducing signaling; etc. The inhibitory agent may inhibit the activity by a variety of different mechanisms. In certain embodiments, the inhibitory agent is one that directly binds to RGMb and, in doing so, inhibits its activity, for example by blocking binding to BMP-2/4.

Representative RGMb inhibitory agents include, but are not limited to: antisense oligonucleotides, antibodies, competitive ligands such as, for example, soluble forms of BMP-2, BMP-4, neogenin, and the like. Other agents of interest include, but are not limited to naturally occurring or synthetic small molecule compounds of interest, which include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing appropriate screening protocols.

An antisense reagent may be antisense oligonucleotides (ODN), particularly synthetic ODN having chemical modifications from native nucleic acids, or nucleic acid constructs that express such antisense molecules as RNA. The antisense sequence is complementary to RGMb, and inhibits its expression. One or a combination of antisense molecules may be administered, where a combination may comprise multiple different sequences. Antisense molecules may be produced by expression of all or a part of the target RGMb sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 25, usually not more than about 23-22 nucleotides in length, where the length is governed by efficiency of inhibition, specificity, including absence of cross-reactivity, and the like. Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature that alter the chemistry of the backbone, sugars or heterocyclic bases.

Anti-sense molecules of interest include antagomir RNAs, e.g. as described by Krutzfeldt et al., herein specifically incorporated by reference. Small interfering double-stranded RNAs (siRNAs) engineered with certain ‘drug-like’ properties such as chemical modifications for stability and cholesterol conjugation for delivery have been shown to achieve therapeutic silencing of an endogenous gene in vivo. To develop a pharmacological approach for silencing RGMbs in vivo, chemically modified, cholesterol-conjugated single-stranded RNA analogues complementary to RGMb mRNA sequences are developed, termed ‘antagomirs’. Antagomir RNAs may be synthesized using standard solid phase oligonucleotide synthesis protocols. The RNAs are conjugated to cholesterol, and may further have a phosphorothioate backbone at one or more positions.

Also of interest in certain embodiments are RNAi agents. In representative embodiments, the RNAi agent targets the precursor molecule of the RGMb mRNA sequence. By RNAi agent is meant an agent that modulates expression by a RNA interference mechanism. The RNAi agents employed in one embodiment of the subject invention are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

In certain embodiments, instead of the RNAi agent being an interfering ribonucleic acid, e.g., an siRNA or shRNA as described above, the RNAi agent may encode an interfering ribonucleic acid, e.g., an shRNA, as described above. In other words, the RNAi agent may be a transcriptional template of the interfering ribonucleic acid. In these embodiments, the transcriptional template is typically a DNA that encodes the interfering ribonucleic acid. The DNA may be present in a vector, where a variety of different vectors are known in the art, e.g., a plasmid vector, a viral vector, etc.

An antagonist of interest for use in the methods described herein includes antibodies that selectively bind to RGMb and inhibit, or block activity or RGMb, including agents that bind to the BMP-2/4 binding site, and/or block the interaction of RGMb with BMP-2/4, neogenin, etc. Antibodies specific for human RGMb include, for example, the rat anti-human antibody BFH-5C9; mouse anti-human antibody (Clone 398528); etc., or antibodies that are generated using art-recognized techniques. Binding to RGMb, and blocking the binding of BMP-2/4 can be tested, for example, by ELISA as described by Xiao et al. (2014), supra.

Antibodies, also referred to as immunoglobulins, conventionally comprise at least one heavy chain and one light, where the amino terminal domain of the heavy and light chains is variable in sequence, hence is commonly referred to as a variable region domain, or a variable heavy (VH) or variable light (VH) domain. The two domains conventionally associate to form a specific binding region, although specific binding can also be obtained with heavy chain only variable sequences, and a variety of non-natural configurations of antibodies are known and used in the art.

A “functional” or “biologically active” antibody or antigen-binding molecule is one capable of exerting one or more of its natural activities in structural, regulatory, biochemical or biophysical events. For example, a functional antibody may have the ability to specifically bind an antigen and the binding may in turn elicit or alter a cellular or molecular event such as signaling transduction or enzymatic activity. A functional antibody may also block ligand activation of a receptor or act as an agonist or antagonist.

The term “antibody” herein is used in the broadest sense and specifically covers monoclonal antibodies, polyclonal antibodies, monomers, dimers, multimers, multispecific antibodies (e.g., bispecific antibodies), heavy chain only antibodies, three chain antibodies, single chain Fv, nanobodies, etc., and also include antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof such as an Fab or F(ab)₂ fragment.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

The antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc.) and human constant region sequences.

An “intact antibody chain” as used herein is one comprising a full length variable region and a full length constant region. An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Antibodies can be derived from any species. In one aspect, the antibody is of largely human origin, or is a humanized version of a non-human antibody. Depending on the amino acid sequence of the constant domain of their heavy chains, intact antibodies can be assigned to different “classes.” There are five major classes of human antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two clearly distinct types, called κ and λ, based on the amino acid sequences of their constant domains.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, an antibody having an Fc region according to this invention can comprise an antibody with or without K447.

“Humanized” forms of non-human (e.g., rodent) antibodies, including single chain antibodies, are chimeric antibodies (including single chain antibodies) that contain minimal sequence derived from non-human immunoglobulin. See, for example, Jones et al, (1986) Nature 321:522-525; Chothia et al (1989) Nature 342:877; Riechmann et al (1992) J. Mol. Biol. 224, 487-499; Foote and Winter, (1992) J. Mol. Biol. 224:487-499; Presta et al (1993) J. Immunol. 151, 2623-2632; Werther et al (1996) J. Immunol. Methods 157:4986-4995; and Presta et al (2001) Thromb. Haemost. 85:379-389. For further details, see U.S. Pat. Nos. 5,225,539; 6,548,640; 6,982,321; 5,585,089; 5,693,761; 6,407,213; Jones et al (1986) Nature, 321:522-525; and Riechmann et al (1988) Nature 332:323-329.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

### Methods

Methods are provided for treating an individual with Inflammatory Bowel Disease (IBD) or chronic pancreatitis, comprising administering an effective amount of an RGMb antagonist to an individual for a period of time sufficient to effect a therapeutic result.

Effective doses of the therapeutic entity of the present invention vary depending upon many different factors, including the means of administration, target site, physiological state of the patient, the severity and course of the disease, the disease being treated (e.g., IBD, chronic pancreatitis, radiation colitis, Crohn's disease, ulcerative colitis, etc.), whether the patient is human or an animal, other medications administered, whether treatment is prophylactic or therapeutic, the patient's clinical history and response to an RGMb antagonist, and the discretion of the attending physician. The an RGMb antagonist is suitably administered to the patient (i.e., the individual) at one time or over a series of treatments.

Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

An exemplary treatment regime entails administration daily, every other day, semi-weekly, weekly, once every two weeks, once a month, etc. In another example, treatment can be given as a continuous infusion. Unit doses are usually administered on multiple occasions. Intervals can also be irregular as indicated by monitoring clinical symptoms. Alternatively, the unit dose can be administered as a sustained release formulation, in which case less frequent administration is required. Dosage and frequency may vary depending on the patient.

The RGMb antagonist composition can be administered parenterally, which includes, but is not limited to intradermal, subcutaneous, intramuscular, intraperitoneal, intravenous, intraarterial, intramyocardial, transendocardial, transepicardial, intrathecal, and infusion techniques. In addition, the RGMb antagonist can be contacted with the donor tissue or cells ex vivo, prior to transplantation.

In an embodiment of the present invention, the composition is administered in an effective amount to decrease, reduce, inhibit or abrogate autoimmune disease related to standard therapy. The amount of antibody in the composition may vary from about 1 ng to about 1 g, more preferably, at least about 0.1 mg/kg, at least about 0.5 mg/kg, at least about 1 mg/kg, at least about 5 mg/kg, up to about 50 mg/kg, up to about 25 mg/kg, up to about 15 mg/kg, up to about 10 mg/kg. In some embodiments the effective dose is from about 0.5 to about 10 mg/kg for an adult human.

Treatment regimens may vary as well, and often depend on the health and age of the patient. Certain types of disease will require more aggressive treatment, while at the same time, certain patients cannot tolerate more taxing regimens. The clinician will be best suited to make such decisions based on the known efficacy and toxicity (if any) of the therapeutic formulations.

An RGMb antagonist can be provided in pharmaceutical compositions suitable for therapeutic use, e.g. for human treatment. In some embodiments, pharmaceutical compositions of the present invention include one or more therapeutic entities of the present invention or pharmaceutically acceptable salts, esters or solvates thereof. In some other embodiments, the use of an RGMb antagonist includes use in combination with another therapeutic agent, e.g., another anti-IBD agent. Therapeutic formulations comprising an RGMb antagonist can be prepared for storage by mixing the RGMb antagonist with optional physiologically acceptable carriers, excipients or stabilizers (Remington's Pharmaceutical Sciences 16th edition, Osol, A. Ed. (1980)), in the form of lyophilized formulations or aqueous solutions. The an RGMb antagonist composition will be formulated, dosed, and administered in a fashion consistent with good medical practice. The “effective amount” of an RGMb antagonist to be administered will be governed by considerations such as those cited above (e.g., severity of disease etc.), and is the minimum amount necessary to prevent and/or reduce the symptoms of the targeted disease (e.g., IBD, chronic pancreatitis, ulcerative colitis, Crohn's disease, radiation colitis, etc.).

Treatment with an RGMb antagonist may be combined with other therapies (including dietary changes, medications and surgery) and an RGMb antagonist need not be, but is optionally formulated with one or more agents that potentiate activity, or that otherwise increase the therapeutic effect. These are generally used in the dosages recommended by the manufacturer, and dosages can readily be optimized. Agents that can be used in combination with an RGMb antagonist include anti-IBD agents and other anti-inflammatory agents.

Examples of suitable anti-IBD agents for combination therapy with an RGMb antagonist include, but are not limited to: 5-aminosalicylic acid (5-ASA); 5-ASA derivatives (e.g., sulfasalazine, mesalamine, balsalazide, olsalazine); antibiotics (e.g., metronidazole, ciprofloxacin, refaximin); corticosteroids (e.g., hydrocortisone, prednisone, methylprednisolone, prednisolone, entocort (budesonide), dexamethasone); immunosuppressants (e.g., azathioprine, 6-mercaptopurine, methotrexate, cyclosporine); DMARDs, TNF inhibitors (e.g., infliximab, adalimumab, certolizumab pegol); monoclonal antibodies (e.g., natalizumab, ustekinumab); histamine H2 antagonists (e.g., cimetidine, ranitidine, famotidine, nizatidine); proton pump inhibitors (e.g., omeprazole, lansoprazole, esomeprazole magnesium, rabeprazole sodium, pantoprazole); antidiarrheals (e.g., diphenoxylate and atropine, loperamide, cholestyramine); anticholinergic, antispasmodic agents (e.g., dicyclomine, hyoscyamine); and the like.

The RGMb antagonist may precede, be co-current with and/or follow the other agent(s) by intervals ranging from minutes to weeks. In embodiments where the composition of the present invention, and other agent(s) are applied separately to a cell, tissue or organism, one would generally ensure that a significant period of time did not expire between the time of each delivery, such that the composition and agent(s) would still be able to exert an advantageously combined effect on the cell, tissue or organism.

An RGMb antagonist is often administered as a pharmaceutical composition as the active therapeutic agent combined with a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

For parenteral administration in an aqueous solution, for example, the solution should be suitably buffered if necessary and the liquid diluent first rendered isotonic with sufficient saline or glucose.

These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The antibodies of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to the compounds formulated for parenteral administration, such as intravenous or intramuscular injection, other pharmaceutically acceptable forms include, e.g. tablets or other solids for oral administration; time release capsules; and any other form currently used.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear an RGMb antagonist by non-covalent associations, such as non-covalent bonding or by encapsulation. The nature of the carrier can be either soluble or insoluble for purposes of the invention. Those skilled in the art will know of other suitable carriers for binding an RGMb antagonist, or will be able to ascertain such, using routine experimentation.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of an RGMb antagonist can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

In some embodiments, the subject methods include monitoring the patient for efficacy of treatment. Monitoring may measure indicia of IBD (e.g. weight loss, colon thickening, soft/loose stool (e.g., diarrhea, watery diarrhea, etc.), rectal bleeding (e.g., bloody stool), abdominal cramps, abdominal pain, vomiting, acute right lower quadrant pain, malaise, fatigue, fever, and/or anemia; etc.) and/or monitoring for the presence or absence (either quantitatively or qualitatively) of a biomarker associated with the disease being treated. For example, diagnosis of IBD and/or chronic pancreatitis (as well as the assessment of treatment efficacy for IBD and/or chronic pancreatitis using the subject methods) can be determined by the presence or absence of biomarkers in a biological sample (e.g., blood, stool, etc.) from the patient followed by colonoscopy and/or any other suitable technique for assessing IBD and/or chronic pancreatitis.

Convenient biomarkers and ways to monitor/measure the biomarkers will be known to one of ordinary skill in the art and any convenient biomarker may be used. Examples of biomarkers that can be used to diagnose and/or determine the severity of IBD (and therefore to monitor the efficacy of the subject methods of treatment for IBD) include, but are not limited to: (i) biomarkers increased in patients with IBD (relative to patients without IBD): C-reactive protein (CRP), ESR, α1 Antitrypsin, α1 antichymotrypsin, α2 macroglobulin, fibrinogen, prothrombin, factor VIIII, plasminogen, tissue plasminogen activator antithrombin, lactoferrin, S100A12, C1s, C2, B, C3, C4, C5, C1INHibitor, C9 Haptoglobin, haemopexin, caeruloplasmin, calprotectin, serum amyloid A, ferritin, Fibronectin, and orosomucoid (α1-acid glycoprotein). Useful antibodies include anti-OmpC, anti-CBir1, anti-I2, anti-A4-Fla2, anti-Fla-X, and antiglycan antibodies; and (ii) biomarkers decreased in patients with IBD (relative to patients without IBD): Factor XII, Albumin, transferrin, Insulin-like growth factor, α-fetoprotein, and cholinesterase.

For examples of IBD biomarkers, see (a) Iskandar et al., Transl Res. 2012 April; 159 (4):313-25: “Biomarkers in Inflammatory Bowel Disease: Current Practices and Recent Advances”; and (b) Vermeire et al., Gut. 2006 March; 55 (3):426-31; both of which are hereby incorporated by reference or their teachings on biomarkers of IBD.

Examples of biomarkers that can be used to diagnose and/or determine the severity of chronic pancreatitis can be found for example, in: Momi et al, Minerva Gastroenterol Dietol. 2012 December; 58 (4):283-97; Jin et al, Intern Med. 2011; 50 (15):1507-16; Paulo et al., Proteomics Clin Appl. 2011 April; 5 (3-4):109-20; Buxbaum et al., JOP. 2010 Nov. 9; 11 (6):536-44; Carroll et al, Am Fam Physician. 2007 May 15; 75 (10):1513-20; Matull et al, J Clin Pathol. 2006 April; 59 (4):340-4; Cavestro et al, JOP. 2005 Jan. 13; 6 (1 Suppl):53-9; and US patent applications 20100184662, 20100144850, 20100099615, and 20050166275; all of which are hereby incorporated by reference for their teachings on biomarkers of chronic pancreatitis.

The improvement is any observable or measurable improvement. Thus, one of skill in the art realizes that a treatment may improve the patient or subjects condition, but may not be a complete cure of the disease. In certain aspects, the composition is administered in an effective amount to decrease, reduce, inhibit or abrogate levels of an immune response against the recipient.

Various combination regimens of the composition and one or more agents are employed. One of skill in the art is aware that the composition of the present invention and agents can be administered in any order or combination.

The invention now being fully described, it will be apparent to one of ordinary skill in the art that various changes and modifications can be made without departing from the spirit or scope of the invention.

### Experimental

### Bone Morphogenetic Protein Signaling Pathway Modulation Promotes Tolerance in Graft-Versus-Host Disease and Prevents Colitis

Neutralization of RGMb signaling using a monoclonal antibody ameliorated immune disorders, such as autoimmunity, in vivo. RGMb expression was significantly increased in murine small intestine at 24 h post total body irradiation (TBI). We evaluated blocking anti-RGMb antibody therapy in inflammatory bowel disease (IBD). Dextran sulphate sodium (DSS) treated-mice were pretreated with anti-RGMb antibody, which resulted in protection of loss body weight for a least 8 days. When DSS-induced colitis mice received pretreatment of the anti-RGMb antibody, survival was increased, as well as normalization of the colon length, unlike with isotype control antibody treated-mice. Blocking anti-RGMb antibody reduces autoimmunity.

It is well known that the mucosa of the gastrointestinal tract is the first line of defense in battling invasion of non-self antigens from commensal microflora and foods, and constitutively active in terms of immunological aspect. This unique environment utilizes an immune system mediated by Th1, Th2, Th17 and Treg cells that play important roles in homeostasis in the intestine through the production of various cytokines. However, imbalance of these Th cell responses in intestine causes inflammatory bowel diseases (IBD) including Crohn's disease (CD) and ulcerative colitis (UC). RBMb expression levels are also high in the gut. The present study investigated the effect of administering a blocking anti-RGMb antibody on experimental colitis induced by dextran sulphate sodium (DSS) in C57BL/6 mice, with the aim to characterize the colonic inflammatory response both at the cellular and molecular levels. The effects and role in the intestinal homeostasis and pathophysiology of IBD were assessed in murine models and in vitro using human naïve T cells.

### Results

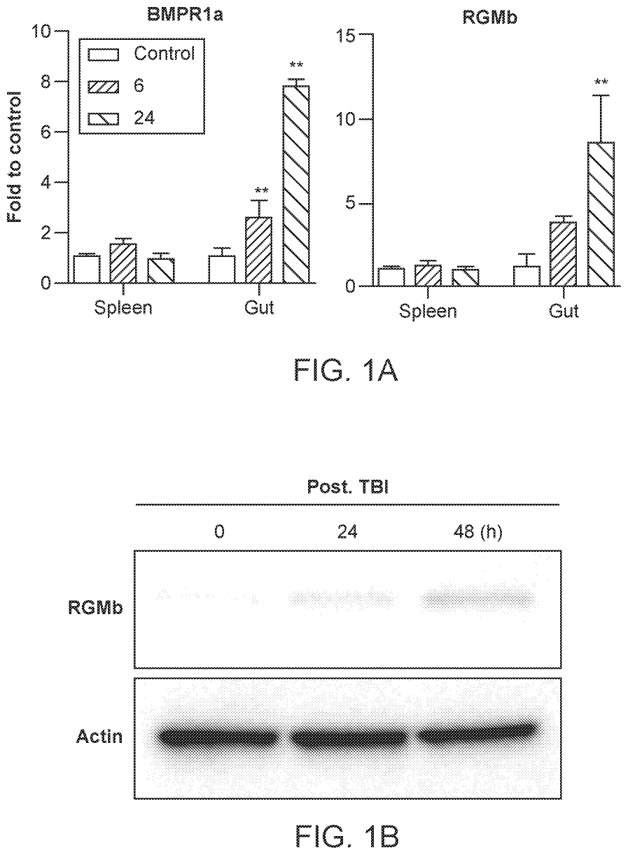

Total Body Irradiation increase RGMb expression in mouse small intestine tissue. RGMb mRNA expression was significantly increased in the small intestine at 24 h post-TBI (p<0.01, FIG. 1A). Induction of RGMb protein expression in the small intestine was observed at 24 h and 48 h post-TBI (FIG. 1B). BMPR1a, the co-receptor of RGMb protein complex, was also significantly increased in the gut at 6 h and 24 h post-TBI (p<0.01, FIG. 1A). BMPR1b receptor is below detection levels in the small intestine. No significant difference in RGMb and BMPR1a expression were found in the spleen post-TBI (FIG. 1A).

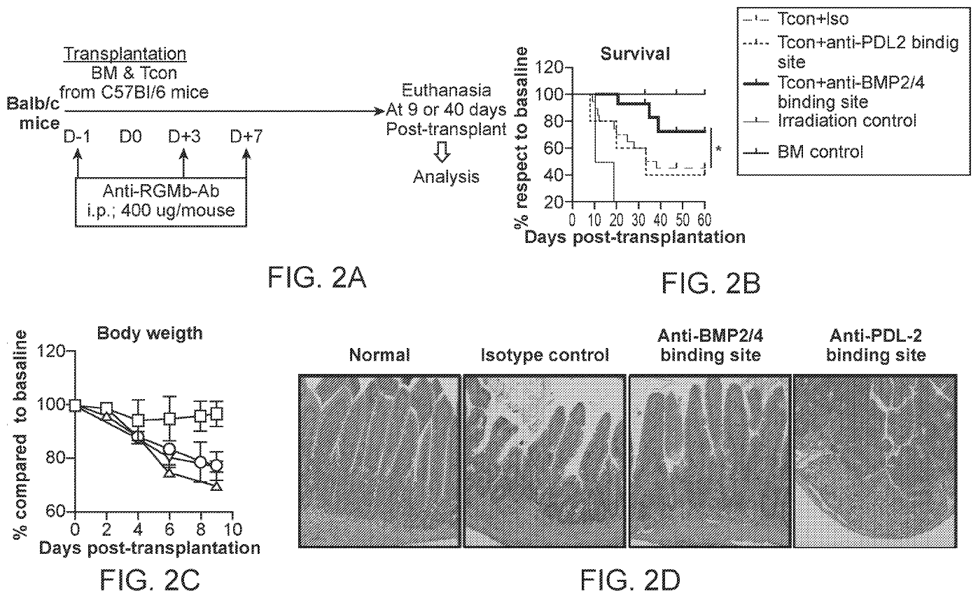

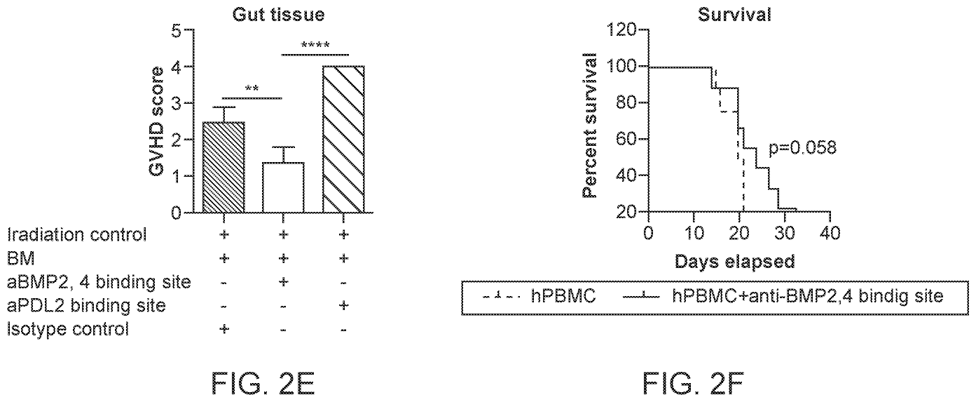

Blocking RGMb using monoclonal antibody protects against GvHD without interfering the Graft-versus-tumor (GVT) effect mediated by allogeneic BMT. An acute GvHD mice model was created by transplantation of T cell depleted bone marrow supplemented with conventional T cells to the recipient mice. To demonstrate the protective effect of blocking RGMb pathway in GvHD induced by allogeneic cells transplantation, anti-BMP2/4 binding site (9D1) and anti-PDL2 binding site (2C9) antibodies were injected into the recipient mice at indicated time points before and after allogenic bone marrow transplantation (FIG. 2A). Treatment with anti-BMP2/4 binding site antibody but not the anti-PDL2 binding site and isotype control antibodies protected the recipient mice against Tcon-induced GvHD with statistically higher body weights achieved and improved survival (75% versus 30% survival rate at 60 days post-transplantation, p<0.05) (FIGS. 2B & 2C).

Previous studies in MHC major mismatch models of acute GvHD have demonstrated that allogeneic T cells infiltration into intestinal area at acute phase of GvHD (9 days post-transplantation) is highly associated with the disease progression in the current murine model. To further investigate how RGMb signaling contributed to GvHD, the recipient mice were sacrificed at 9 days post-transplantation and intestinal tissues were harvested. Histopathological analysis of mouse ileum showed that treatment with anti-RGMb antibody markedly decreased lesions targeting blood vessels and small intestinal villi and reduced the GvHD histology score measured by a blinded pathologist (P<0.05) in the gut of GvHD mice compared to control mice (FIGS. 2D & 2E). The translational application of anti-RGMb therapy was further demonstrated by using human PBMCs transplanted in NSG mice. PBMCs survival was monitored during the 34 days post-transplantation and blocking RGMb signaling significantly protected the transplanted human PBMCs (p<0.05, FIG. 2F). In addition, anti-RGMb therapy significative delay human T cell maturation in vitro after 7 days of mixed lymphocyte reaction with CD11b⁺ cells (p<0.039). Together, the results showed that anti-RGMb therapy was effective on both mouse and human donor cells.

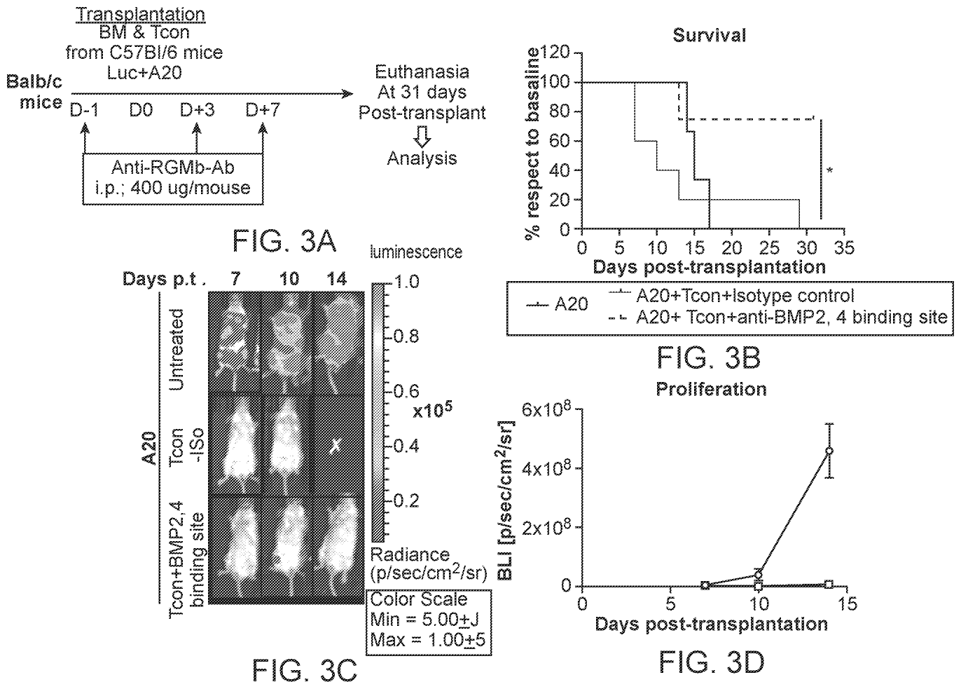

We further examined if anti-RGMb could reduce GvHD without interfering the therapeutic GVT effect mediated by bone marrow transplantation. To test this hypothesis, murine B cell lymphoma (A20) cells carrying luciferase reporter gene were inoculated together with allogeneic BMT, and the recipient mice were treated with anti-RGMb or isotype control antibodies at indicated time points (FIG. 3A). The recipient mice without allogeneic BMT all died from leukemia at day 17 after inoculation of A20 cells. Transplantation of Tcon cells plus anti-RGMb treatment, but not isotype control, significantly improved the survival rate in the recipient mice (p<0.05, FIG. 3B). By tracking the leukemia with luciferase signaling in vivo, we found that allogeneic BMT have mediated the clearance of inoculated leukemia in both anti-RGMb or isotype control treated mice (FIGS. 3C & 3D). Anti-RGMb therapy was able to reduce GvHD without interfering the beneficial GVL effect mediated by allogeneic BMT. Comparatively, isotype control failed to protect the recipient mice due to GvHD, though the implanted leukemia was cleared.

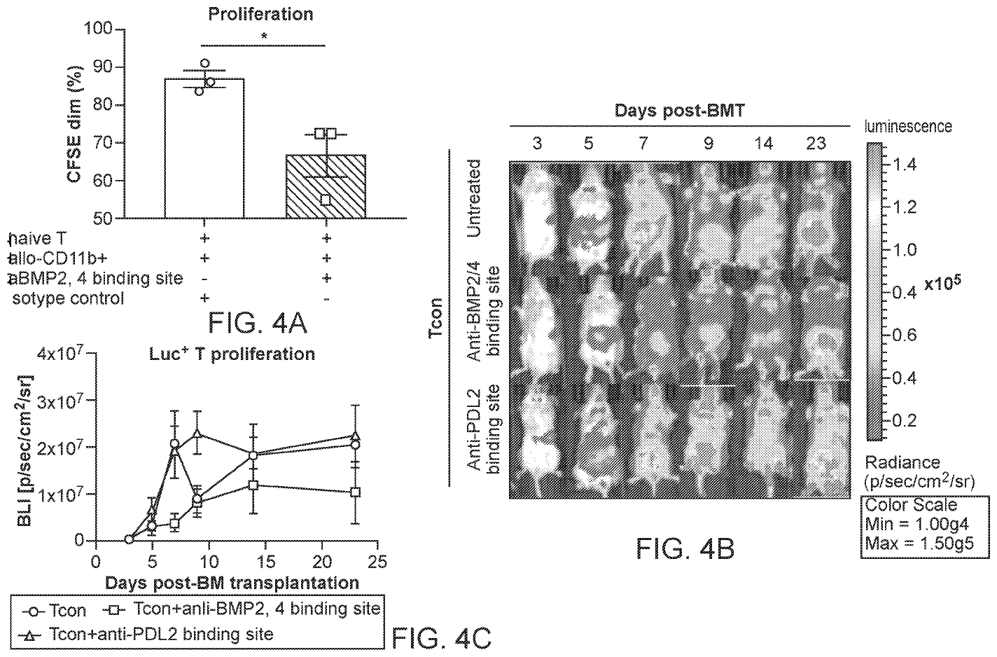

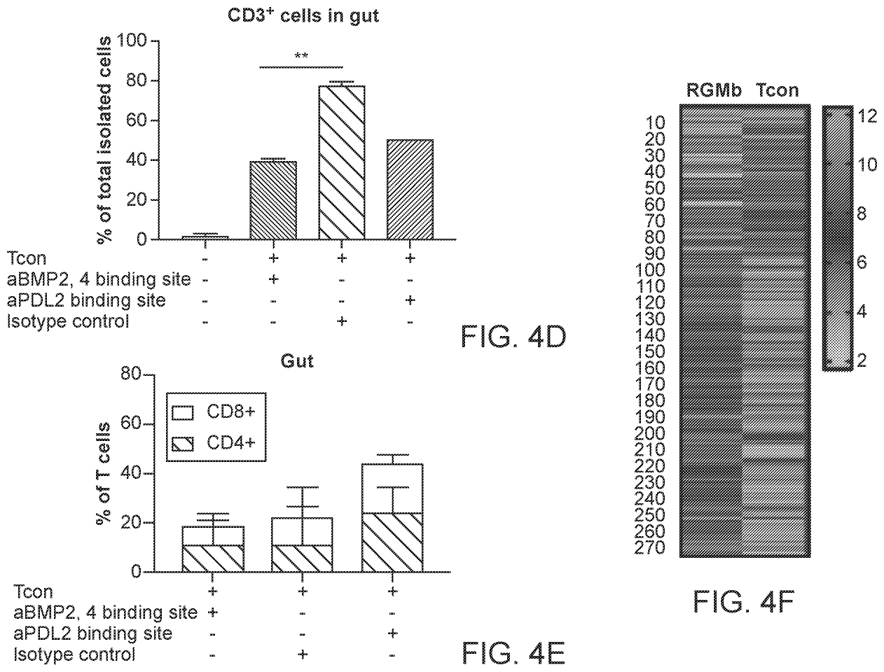

Blocking RGMb using monoclonal antibody reduced T cell proliferation in vivo and in vitro. To further characterize how anti-RGMb therapy reduced CD3⁺ T cell infiltration into small intestine, allogeneic BMT with Tcon expressed luciferase gene were used to monitor the transplanted Tcon trafficking in vivo. The treatment of monoclonal anti-PDL2 antibody (2C9), which blocks RGMb/PDL2 interaction without disrupting RGMb/BMP2,4 binding, was included to further dissect the mechanism of anti-RGMb therapy. Consistent with the result in FIG. 2B, anti-RGMb therapy protected the recipient mice against allogeneic BMT associated GvHD. However, anti-PDL2 therapy failed to protect the recipient mice against GvHD and further reduced the survival rate. Bioluminescence imaging study showed that anti-BMP2,4, but not anti-PDL2 binding site antibody, reduced T cell proliferation in vivo (FIG. 4A).

Quantification results indicated that anti-RGMb therapy significantly reduced T cell proliferation at day 6, while anti-PDL2 therapy increased the signal at day 9, compared to the isotype control treated group (FIG. 4A).

Small intestine tissues from the recipient mice were digested into single cell suspension to quantify the infiltrated T cells using flow cytometry. Increased CD3⁺ T cells were found in gut of GvHD mice treated with isotype control, which was reduced in the anti-BMP2,4 binding site treated group (p<0.05, FIG. 4B). In addition, spleen CD8⁺/CD4⁺ T cell ratio was increased in GvHD mice compared to BM transplantation control. Interestingly, treatment of anti-BMP2,4 binding site antibody significantly reduced CD8⁺/CD4⁺ ratio in spleen but not in gut (p<0.05 FIGS. 4C & 4D).

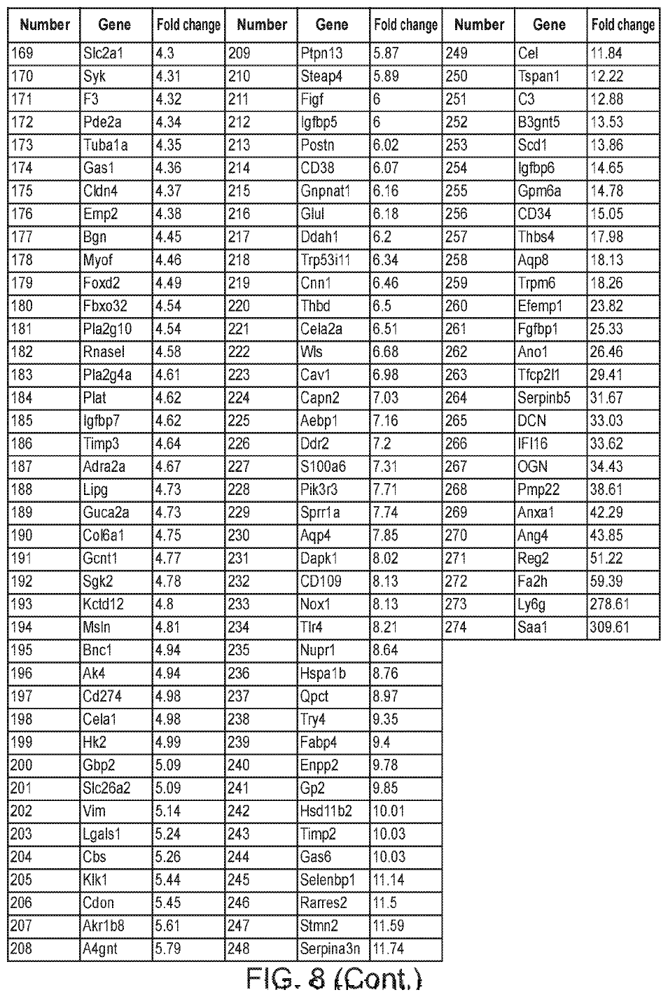

A heat map was generated from DNA microarray data, reflecting gene expression values in gut after 9 days of BM transplantation (FIG. 4E). T cells maturation and proliferation gene expression significantly decreased after anti-RGMb treatment in gut. CD109, CD274, CCL25, Foxq1 and Tm4SF4 are relevant examples of these genes. CD34, a molecule expressed in hematopoietic progenitor cells, was increased. Moreover, Mertk, a gene related to cytokine signaling, was decreased after anti-RGMb treatment (FIG. 8).

Anti-RGMb antibody treatment reduces inflammation following GvHD in mice. Since Th1 and Th17 cytokines play a key role in the control of gut inflammation, we analyzed gene expression by qPCR in mouse gut. 9 days post-BMT showed increased IL-4, IL-17 and INF-γ expression in gut lysates of mice. Significantly lower expression of IFN-γ was detected in gut tissue lysates from anti-RGMb and BM-transplanted mice, compared to control animals. IL-4, IL-10 and IL-17 gene expression did not shown significant difference between anti-RGMb treated mice and controls. In addition, Neogenin, BMPRIa and BMP2, BMP4 genes do not show a significant difference in mouse gut after treatments.

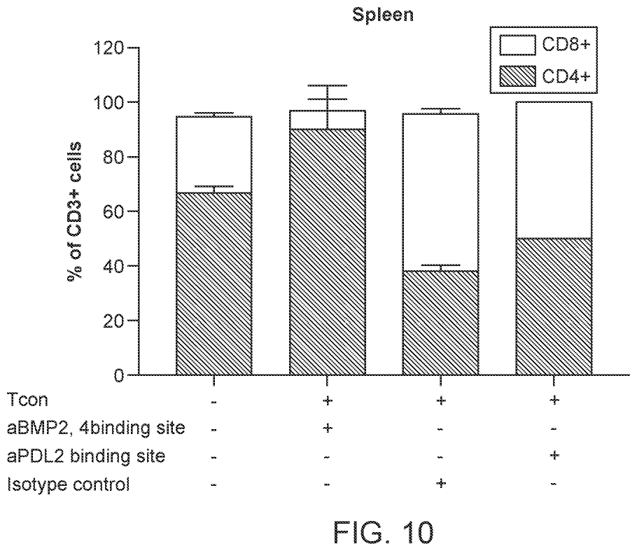

Cytokine production were measured ex vivo after CD3-CD28 beads (Gibco™) activation of splenocytes from anti-BMP2,4 antibody treated-mice, compared to the control isotype at 9 days BMT (FIG. 10). Anti-BMP2,4-treated mice showed levels of IL-5, IL-13, IL-17 and VEGF significantly lower than the isotype control mice, whereas the levels of GM-CSF were higher in these animals (p≤0.05). No significant difference were observed in IL-1b, IL-6, IL-12, MIP1a, MCP1, MIG, IP10 levels after RGMb treatment compare to controls. Levels of FGFb, IL-10, IL-1a, KC were undetectable with this protocol.

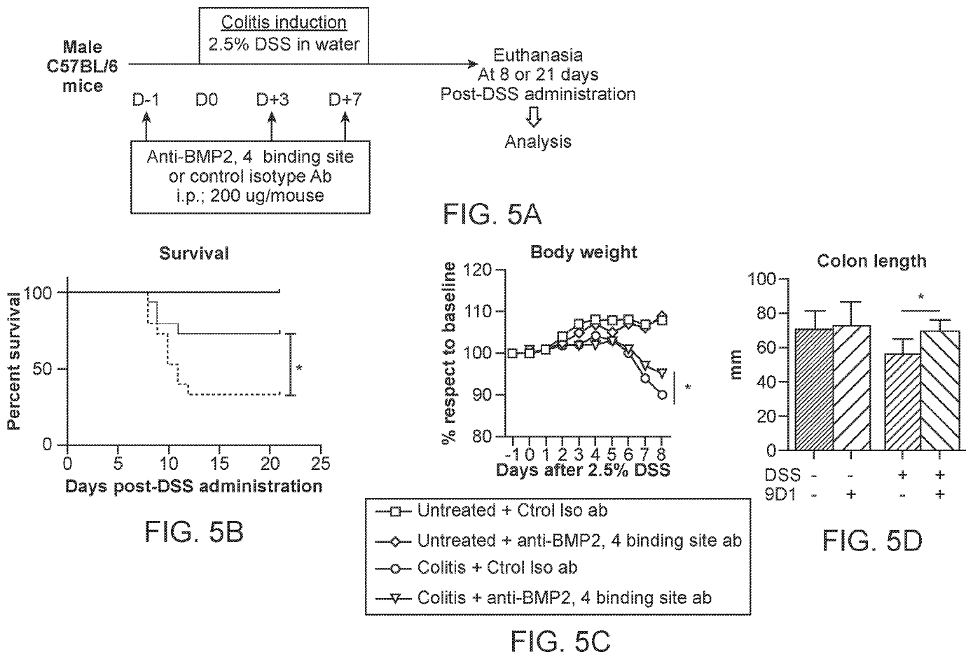

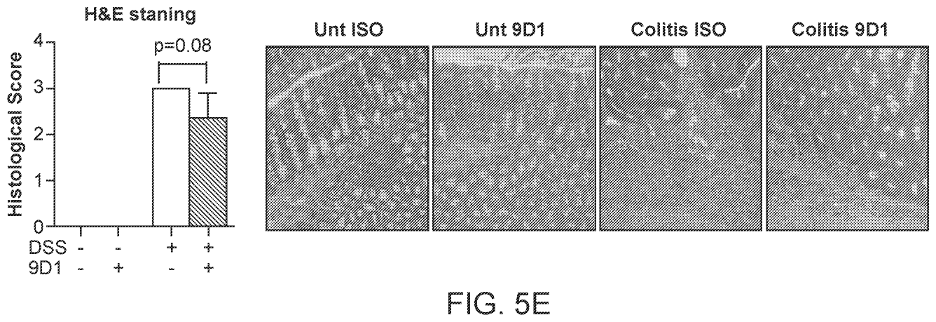

Treatment with anti-RGMb antibody prevent inflammatory bowel diseases in mice. Treatment with monoclonal anti-RGMb antibody, which blocks BMP2/4 binding site interaction without disrupting RGMb/BMP binding, was included to assess the impact of Bone Morphogenetic Protein signaling pathway modulation in inflammatory bowel diseases. Male C57BL/6 mice were injected with anti-RGMb or isotype control antibody (200 μg/mouse; i.p.). Mice received 3 doses of antibodies. 24 h after the first injection, mice were treated or not with 2.5% DSS in their drinking water for 7 days to induce colitis (FIG. 5A). Mortality was monitored up to the 21^{st} day.

DSS treated mice showed that anti-RGMb therapy protected the recipient mice against colitis (FIG. 5B). Control isotype antibody therapy failed to protect the recipient mice against colitis and further reduced the survival rate (FIG. 5B). DSS untreated mice increased their body weight during the 8 days, in contrast body weight was significantly decreased in mice after DSS administration. Anti-RGMb antibody treatment significantly reduced body weight loss (FIG. 5C). As shown in FIG. 5D, the colon lengths were similar among untreated animals, but DSS treatment led to a significant reduction in length for isotype control mice, which was not evident after anti-RGMb treatment.

Histopathological analysis of gut showed that anti-RMGb antibody treatment in colitis mice significantly reduced inflammation and tissue remodeling compared to the isotype control treated mice. Treatment with anti-RMGb antibody had no negative effect in inflammatory cell recruitment in the gut of untreated DSS mice at 9 days compared to control isotype treated mice (FIG. 5E). This was confirmed by the histopathological score evaluating both inflammation and gut tissue remodeling (2.3±0.67 vs 1.7±0.67 in control isotype- and anti-RGMb-treated colitis mice, respectively, p<0.05).

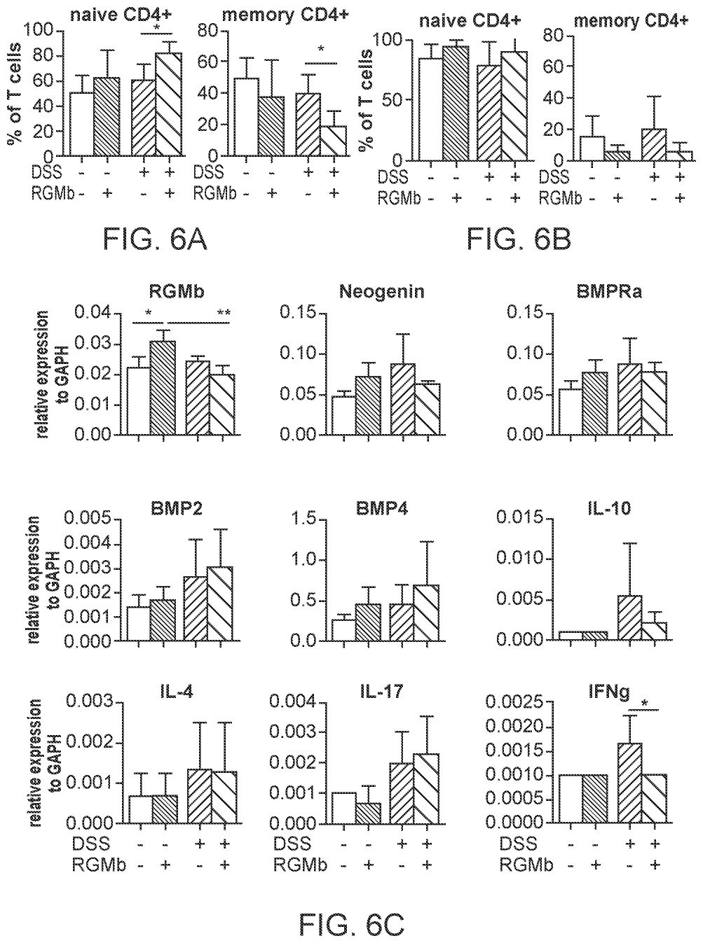

Anti-RGMb antibody treatment reduces the inflammation following inflammatory bowel diseases in mice. Colitis is associated with enhanced gut inflammation relative to control mice, so we next characterized inflammatory and immune cells in the spleen and gut of each group. At 9 days post-DSS administration, we observed an increased naïve CD4⁺ cell number in the spleen but not in gut of DSS-treated mice, compared to controls (FIG. 6A). Effector memory CD4⁺ cell number was decreased in the spleen and gut of colitic mice (FIGS. 6A & 6B).

Th1 and Th17 cytokines play a key role in the control of gut inflammation, therefore we analyzed gene expression by qPCR in mouse gut (FIG. 6C) 9 days post-DSS administration. There were increased IL-4, IL-17 and INF-γ expression in gut lysates of mice. Significantly lower expression of IFN-γ was detected in gut tissue lysates from anti-RGMb and DSS-treated mice compared to control animals. IL-4, IL-10 and IL-17 gene expression did not show significant difference between anti-RGMb treated mice and controls. Neogenin, BMPRIa and BMP2, BMP4 genes did not show a significant difference in mouse gut after treatments.

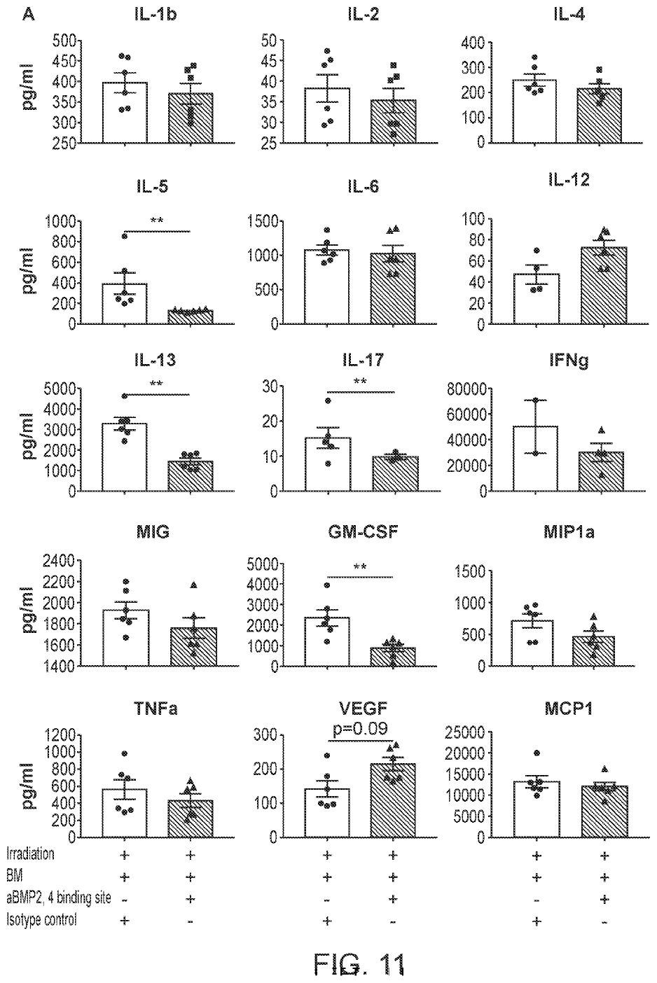

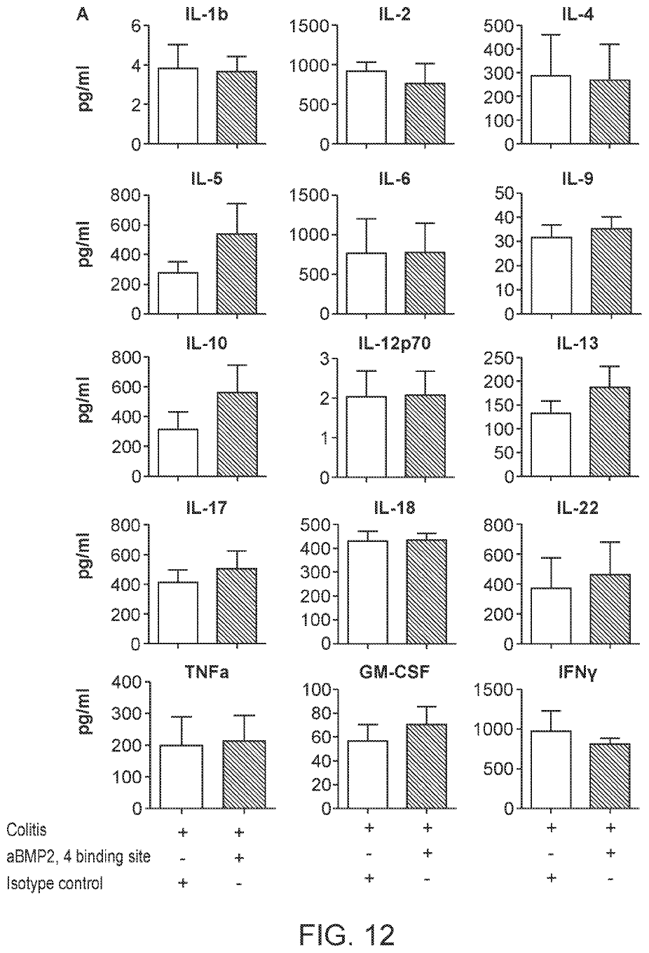

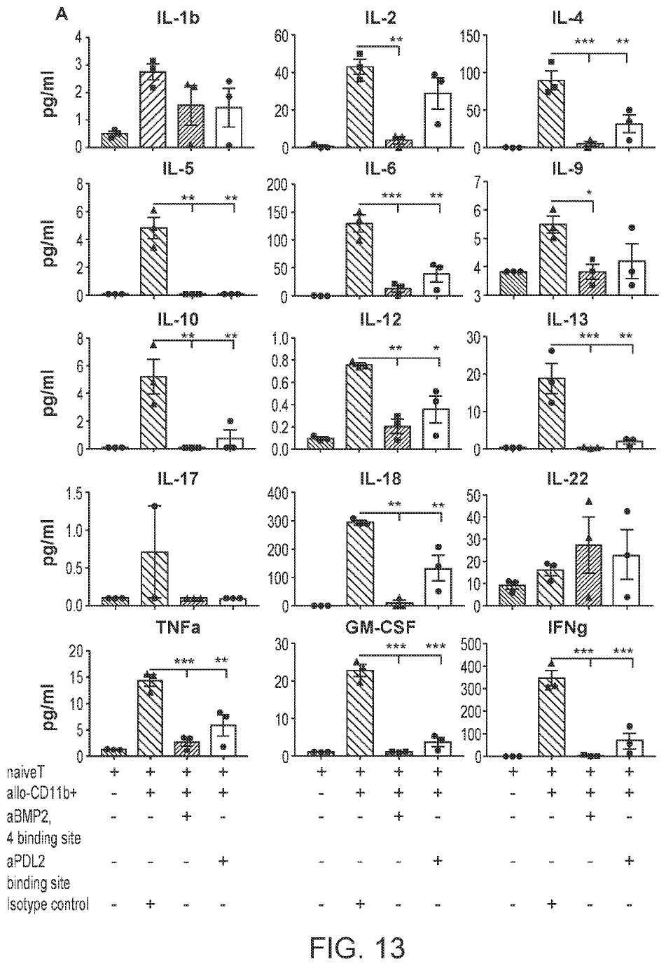

Cytokine production were measured ex vivo after CD3-CD28 beads (Gibco™) activation of splenocytes from anti-BMP2,4 antibody treated—compared to the control isotype mice at 9 days DSS-administration (FIG. 11). Anti-BMP2,4-treated mice showed levels of IL-5, IL-13, IL-17 and VEGF that were significantly lower than the isotype control mice, whereas the levels of GM-CSF were higher in these animals (p≤0.05). No significant difference were observed in IL-1b, IL-6, IL-12, MIP1a, MCP1, MIG, IP10 levels after RGMb treatment compared to controls. Levels of FGFb, IL-10, IL-1a, KC were undetectable with this protocol.

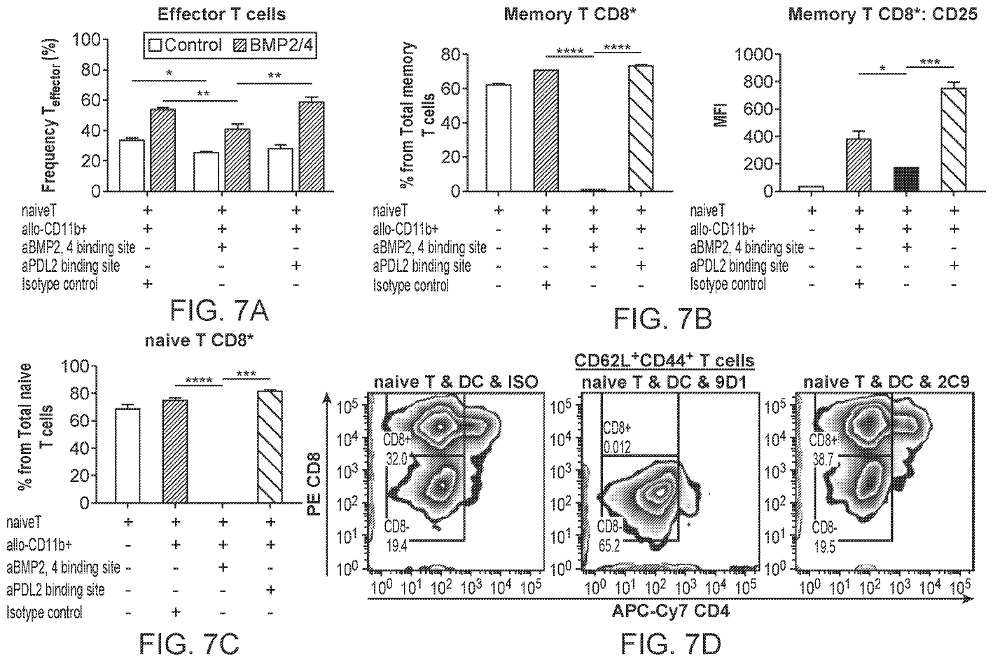

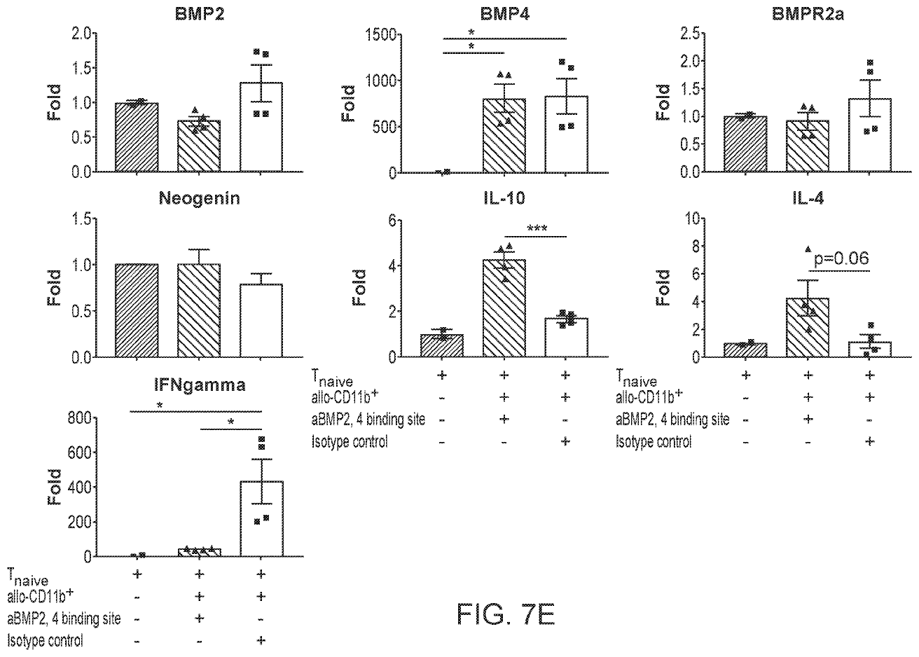

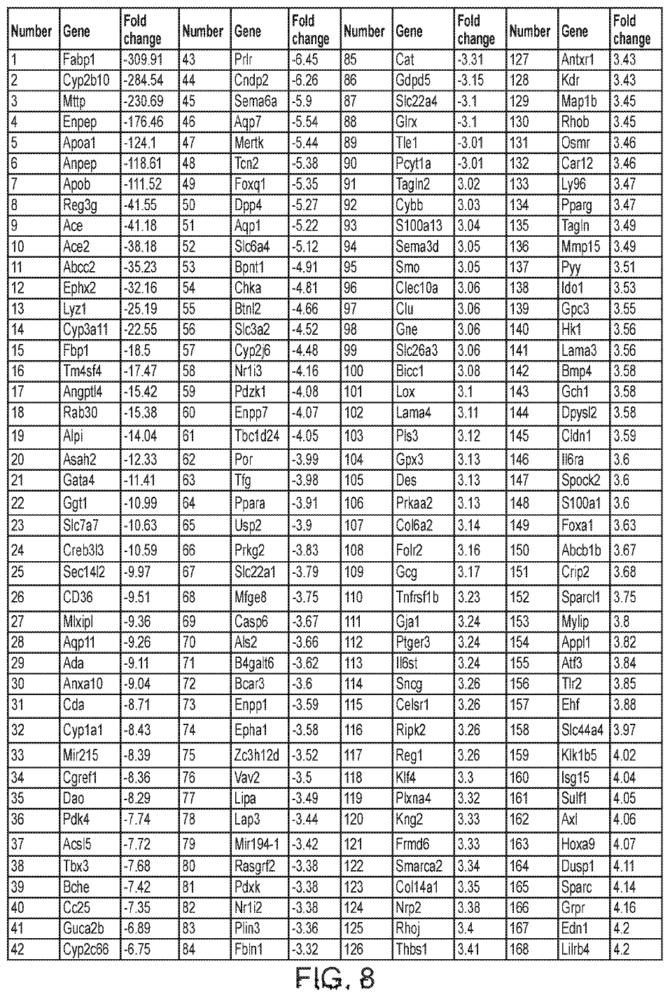

Anti-RGMb antibody therapy delayed T cell maturation. The phenotype was further confirmed in vitro that anti-RGMb treatment reduced both naïve CD4⁺ and naïve CD8⁺ T cell proliferation in the mixed lymphocyte reaction assay (FIG. 7). Anti-PDL2 treatment, however, increased naïve CD4⁺ T cell proliferation and had no effect on naïve CD8⁺ T cell proliferation. Together, the results indicated that interruption of the binding between RGMb/BMP/neogenin, but not RGMb/PDL2, is crucial for the protective effect in anti-RGMb therapy.

In addition, allogeneic Tcon transplantation induced inflammatory cytokine expressions (IFN-γ, BMP2, and BMP4) in small intestine tissue, whereas anti-RGMb therapy enhanced the anti-inflammatory response via increased IL-10 and decreased IFN-γ and BMP2 expression (FIG. 7). Taken together, these data demonstrate that the protective effect of anti-RGMb antibody treatment was mediated via reduced CD3⁺ T cell infiltrated and inflammatory cytokine secretion in small intestine, reduced CD8⁺ in spleen, and alleviated IBD, including colitis, as well as GvHD induced intestinal tissue damage in mice.

RGMb expression was originally discovered in the neuron system associated with neuronal cell differentiation, migration, and apoptosis. Expression has now been identified in several other tissues and organs including skeletal, gut, and immune system. In murine gut tissue, RGMa and RGMb, but not RGMc, was detected in enteric ganglia cells and intestinal epithelium, predominantly in the proliferative crypt compartment. Here we report that a regimen of TBI further enhanced RGMb expression in intestinal epithelium. This phenomenon may increase the interaction of intestinal epithelium with infiltrating lymphocytes, which can be blocked by the anti-RGMb antibody treatment.

Dutt et al. reported that naïve CD4⁺ T cells expanded in mesenteric lymph node and gut after transplantation, and induced severe colitis during the pathogenesis of GVHD (Dutt, S. et al. 2007). In our current findings, blocking the RGMb with monoclonal antibody successfully inhibited T cell proliferation in a mixed lymphocyte reaction assay (FIG. 4E) but failed to interfere the proliferation directly induced by BMP2/4 or CD3/CD28, indicating that RGMb signaling is only involved in T cell activation induced by antigen presenting cells. In addition, RGMb-mediated T cell activation was also found in the mixed lymphocyte reaction with other antigen presenting cells, suggesting that the RGMb expression may not be limited to the CD11b⁺ macrophages.

After transplantation, the interaction between donor T cells and recipient antigen presenting cells induces T cell activation and proliferation. Our data demonstrated the therapeutic effects of anti-RGMb therapy. Anti-RGMb therapy suppressed initial T cell activation based on the in vitro mixed lymphocyte reaction assay (FIG. 4E) and in vivo donor T cell proliferation in the gut at day 9 post-transplantation (FIG. 4B). Inhibition of RGMb signaling in gut tissue enhanced by TBI may alleviate acute inflammation in response to local tissue damage, and consequently reduce T cell infiltration. Anti-RGMb therapy reduced the IFNγ secretion and T cell mediated cytotoxicity in the gut tissue. Notably, we observed that the suppression of T cell proliferation by anti-RGMb treatment was more extensive in CD8⁺ T cells compare to CD4⁺ T cells (FIG. 4).

Importantly, our data showed that transplanted T cells remained competent to mediate GVT effect during anti-RGMb therapy. Although the leukemia cells was detected at earlier time points in the lymph node from mice with anti-RGMb therapy (FIG. 3C), the complete clearance of leukemia cells was observed later.

In conclusion, these data demonstrate the potent immunosuppressive effects of anti-RGMb Ig against IBD, including colitis. The blocking antibody-based inhibition of bone morphogenetic protein signaling pathways through RGMb provides a unique strategy for immunosuppression.

### Materials and Methods

Mice. Eight-weeks old BALB/CJ, NSG (NOD.Cg-Prkdc^{scid} II2rg^{tm1Wjl}/SzJ), C5BL/6 mice were purchased from Jackson Laboratories (Sacramento, Calif.). Luciferase-expressing (luc⁺) C57BL/6 mice were created as described previously [Zeiser et al., 2008]. Mice were maintained under a 12-hours light-dark cycle and they were fed with a standard laboratory diet. The inhalational anesthetic isoflurane was administered during bioluminescence imaging (BLI). All studies were approved by Institutional Animal Care and Use Committee of Stanford University.

Bone marrow and cell transplantation. BALB/c mice were conditioned with total body irradiation (2×400 cGy, 200 kV X-ray source; Kimtron), injected with 5×10⁶ T-cell depleted donor bone marrow (TCD-BM) cells combined with 1×10⁶ CD4⁺/CD8⁺ conventional T cells (Tcons) from C57BL/6 or luc⁺ C57BL/6 mice. Luciferase-expressing mouse B cell lymphoma cell line A20 (ATTC) were used to induce leukemia. 1×10⁴ of luc⁺A20 cells was injected at the moment of bone marrow transplantation by intravenous route.

NSG (NOD-SCID IL2 receptor gamma null) mice received 250 cGy total body irradiation (TBI) before injection. Human PMBC (7×10⁷ cells/mouse) were injected into the tail vein to create graft-versus-host disease. Anti-RGMb (9D1) or isotype control antibody were given at indicated time points.

Cell isolation and flow cytometry. Tcons were prepared from C57BL/6 splenocytes and lymph nodes and enriched with CD4 and CD8 MicroBeads (Miltenyi Biotec). TCD-BM cells were prepared by flushing murine tibiae and femora with PBS supplemented with 2% FCS followed by depleting T cells with CD4 and CD8 MicroBeads (Miltenyi Biotec) reaching a purity>99%.

The isolated splenocytes and single gut cells after collagenase digestion were washed twice in staining buffer consisting of phosphate-buffered saline supplemented with 1% fetal bovine serum. Flow cytometry followed routine procedures using 1×10⁵ cells per sample. Cells were labeled with fluorescein isothiocyanate—PE-CD4, CD8, CD25, CD69, CTLA4, Lag3 and PD1 antibody, (Becton Dickinson, San Diego, Calif.) to measure the expression. Flow-cytometric analysis was conducted on a FACS LSR II (Becton Dickinson) and analyzed using the FlowJo analysis program v7.6.5. Gating strategies are reported by Sharan and colaborators.

Mixed lymphocyte reaction. CD11b⁺ cells were isolated from human PBMCs or C57BI/6 mice and were incubated with FACS-purified human naïve T cells (CD45RA⁺, CCR7⁺) or naïve T cells from BALB/c mice (TCR⁺CD62L⁺CD44⁻). Mixed cells were cultured for 7 days in RPMI 1640 supplemented with 2 mmol/L L-glutamine, 1 mmol/L sodium pyruvate, 100 U/mL penicillin, 100 μg/mL streptomycin, and 10% FCS (Gibco®). The naïve T and CD11b⁺ cell ratio was 5:1. Human T cells were labeled with Aqua, CD4 [RPA-T4], CD8 [RPA-T8], CD3 [OKT3], CCR7 [G043H7], PD1 [EH12.2H7] (Biolegend, San Diego, Calif.), CD45Ra [HI100] (eBioscience, San Diego, Calif.), and CD25 [M-A251] antibody (BD Biosciences, San Jose, Calif.) and measure the expression. In the other hand, mouse T cells were labeled with Aqua, CD4 [RM4-5], CD8 [53-6.7], CD3 [17A2], CD44 [IM7], CD62L [MEL-14], (Biolegend, San Diego, Calif.), PD1 [J43], TCR [H57-597] (BD Bioscience, San Jose, Calif.), CD25 [PC61.5] (Invitrogen) and CD45 [30-F11] antibody (Tombo biosciences), to measure the expression. Cytokines profile in supernatant were analyzed using human or mouse ultrasensitive cytokine magnetic 10-Plex panels (Invitrogen).

Induction of Acute DSS-Induced Colitis. Colitis was induced by the administration of DSS (molecular weight, 40 kilodaltons; Sigma Aldrich). 2.5% DSS were dissolved in sterile, distilled water ad libitum for 7 days. Fresh DSS solution was prepared daily followed by normal drinking water. Mice were sacrificed at day 8 following colitis induction (Wirtz S et al. 2007; Montbarbon M et al., 2013). Control animals were either untreated. To survival experiment, animals were monitored daily for behavior, aspect alteration and body weight loss for a period of 21 days. Animals presenting signs of suffering (weight loss>20%, prostration, tremors) were instantaneously euthanized by cervical dislocation. The entire colon was removed from the caecum to the anus, then measured. Anti-RGMB (9D1) or isotype control antibody were given at indicated time points (FIG. 5A).

Cytokine measurement. Levels of IFN-γ, IL-1β, IL-2, IL-4, IL-10, IL-6, and tumor necrosis factor alpha (TNF-α) were quantified in sera, using commercial Luminex kits (Invitrogen, Minneapolis, USA). Similarly, levels of human IFN-γ, IL-1β, IL-2, IL-4, IL-10, IL-6, and tumor necrosis factor alpha (TNF-α) were measured in the supernatants of mixed lymphocyte reaction after 7 days. 0.5×10⁶ of splenocytes by well were re-stimulated in vitro with CD3-CD28 beads (Gibco®) during 72 h.

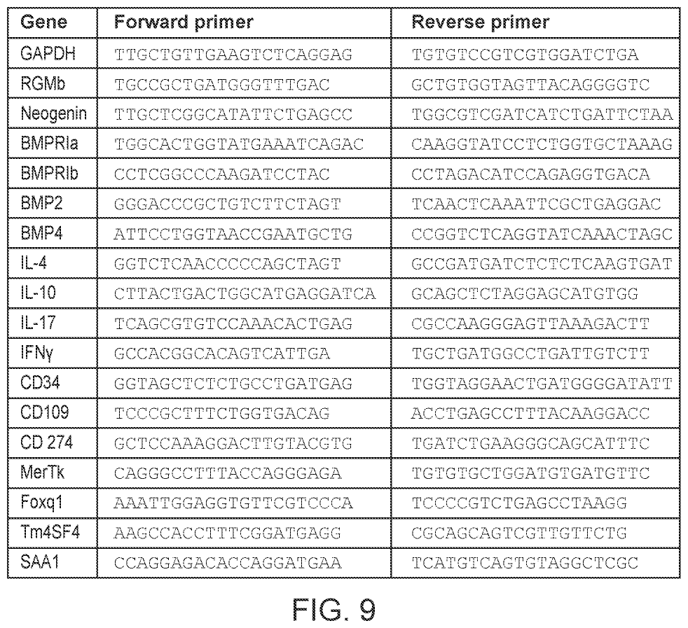

RNA extraction, quantitative real-time PCR and gene expression microarray. Total RNA was isolated from the small intestine of mice using the RNeasy mini kit (QIAGEN). First-strand cDNA synthesis was performed using the SuperScript II kit (Life Technologies) and amplified using the Bio-Rad qPCR System (Bio-Rad) using specific primers for mouse RGMb, neogenin, BMPRIIa, TNF-alpha, BMP4, IL-10 (FIG. 9). Gene expression microarray was performed as previously described (Zhuang et al. 2015; Li et al., 2016; Li et al., 2018). Briefly, Cy3-labeled cRNA for microarray hybridization was prepared with the One-Color Low Input Quick Amp Labeling Kit (Agilent, USA) and then purified using the RNeasy Mini Kit (Qiagen, Germany). Cy3-labeled cRNA was fragmented and hybridized to the array for 17 h at 65° C. in a rotating Agilent hybridization oven. After hybridization, arrays were washed and dried, then scanned immediately on the Agilent Microarray Scanner. Intensity values of each scanned slide were extracted using Agilent Feature Extraction software (version 10.7.3.1; Agilent Technologies).

Gene expression analysis. Gut tissue was collected into a RNA stabilization buffer (Ambion) and was cut into small pieces and homogenized before loaded onto a QIAshredder spin column (Qiagen). Raw data analyses were performed with GeneSpring GX software (Version 12.0; Agilent Technologies). The intensity values were log 2 transformed by quantile normalization. The Welch t test (p values) was applied to identify differentially expressed genes in NOA compared to OA. The p values were corrected by the false discovery rate of Benjamini and Hochberg (q values) analyses. Fold change (FC) values were calculated for each gene as the difference between the mean intensity of the NOA samples and mean intensity of the OA samples. Genes with an FC value>2 or <½ and a q value<0.05 were considered to be differentially expressed. Quality control analysis of microarray gene expression data was performed as previously described.

Microarrays. Total RNA was extracted, using a RNeasy Micro Kit (Qiagen) and its quality was assessed by and Agilent 2100 Bioanalyzer. The list of 969 differentially expressed genes (Fold change>3) was uploaded onto the website of Ingenuity Pathway Analysis. Each gene was mapped to its corresponding gene object in the Ingenuity Pathways Knowledge Base and biological function categories of each gene was annotated by Ingenuity Pathway Analysis (Qiagen). Total RNA was extracted, using a RNeasy Micro Kit (Qiagen) and its quality was assessed by and Agilent 2100 Bioanalyzer. Total RNA (from 500 pg-2 ng) was reverse transcribed into cDNA followed by in vitro transcription use GeneChip™ WT Pico (Affymetrix, Santa Clara, Calif.). Labeled cRNAs were hybridized to (Affymetrix) according to the manufacturer's protocol, and the chips were scanned using a GeneChip Scanner 3000 GeneChip™ Mouse Gene 2.0ST—this is a whole transcript design with probes at 3′ as well as exon. Catalog number: 902118 (Affymetrix). Background correction, normalization and estimation of gene expression was performed using the Robust Multiarray Average (RMA) method in R/Bioconductor. Following aggregation, the samples were re-normalized using quantile normalization.

Primers. Quantitative RT-PCR was performed to quantify mRNA of interest (FIG. 9). Results were expressed as mean±SEM of the relative gene expression calculated for each.

Western Blotting. Mouse small intestine were lysed in RPIA lysis buffer (Invitrogen) containing protease inhibitor mixture (Roche) for 30 min on ice. After centrifugation for 10 min at 4° C., the supernatant was assayed for protein concentration by colorimetric assay (BCA kit, Pierce). The lysates were subjected to Western blotting analysis using anti-RGMb antibody (abcam, ab-96727) and anti-β-actin antibodies (Santa Cruz, sc-47778) as indicated.

Immunohistochemistry staining. Intestinal tissues were removed, its length measured and fixed in formalin. Five-micrometer slides were cut longitudinally and stained with hematoxylin and eosin [H&E] using a standard protocol. Colitis model slides were scored for tissue quality [poor or moderate to perfect]. Based on the existing literature, eight histological components were assessed: inflammatory infiltrate, goblet cell loss, hyperplasia, crypt density, muscle thickness, submucosal infiltration, ulcerations and crypt abscesses [all categorized from 0-3, Koelink P J et al. 2018) J Crohns Colitis. 12 (7):794-803]. A total histological severity score, ranging from 0 to 24, was obtained by summing the eight item scores. GvHD model histopathology was scored as described in Schneidawind D et al. 2014 Blood. 2014; 124 (22):3320-3328.

In vivo bioluminescence imaging. BLI was performed as described previously (Xenogen) Briefly, firefly luciferin (Biosynth) was injected intraperitoneally 10 min prior to image acquisition with an IVIS spectrum imaging system (Xenogen). Images were analyzed with Living Image Software 4.2 (Xenogen).

Sample size and statistical analysis. GraphPad Prism statics software (GraphPad Software Inc., San Diego, Calif., USA) was used for analysis and data graphing. Results were analyzed using non-parametric test (Mann Whitney tests), T test expressed in terms of probability (P). Differences were considered significant when P<0.05. All data are expressed as mean±SEM. experiment in folds (2-ΔΔCt) using GAHP as a reference.

1. A method for treating a chronic inflammatory gastrointestinal disease, the method comprising:
administering to said subject a therapeutically effective amount of an antagonist of RGMb.

2. The method of claim 1, wherein the antagonist is an antibody.

3. The method of claim 2, wherein the antibody specifically binds to RGMb and blocks the interaction between RGMb and BMP-2/4 proteins.

4. The method of claim 1, wherein the chronic inflammatory gastrointestinal disease is inflammatory bowel disease (IBD).

5. The method of claim 4, wherein the IBD is Crohn's disease.

6. The method of claim 4, wherein the IBD is ulcerative colitis.

7. The method of claim 4, wherein the IBD is radiation colitis.

8. The method of claim 1, wherein the individual is a mammal.

9. The method of claim 8, wherein the individual is a human.

10. The method of claim 1, wherein the individual is suspected of having Inflammatory Bowel Disease (IBD).

11. The method of claim 1, wherein the individual has been diagnosed as having Inflammatory Bowel Disease (IBD).

12. The method of claim 4, wherein the effective amount is effective at reducing at least one symptom associated with IBD selected from the group consisting of: weight loss, colon shortening, soft stool, diarrhea, bloody stool, abdominal cramps, abdominal pain, vomiting, acute right lower quadrant pain, malaise, fatigue, fever, and anemia.

13. The method of claim 4, wherein an RGMb antagonist is administered in combination with:
(i) an anti-IBD agent selected from the group consisting of: 5-aminosalicylic acid (5-ASA), a 5-ASA derivative, an antibiotic, a corticosteroid, an immunosuppressant, a TNF (tumor necrosis factor) inhibitor, natalizumab, ustekinumab, a histamine H2 antagonist, a proton pump inhibitor, an antidiarrheal, and an anticholinergic.

14. The method of claim 4, further comprising monitoring the individual for symptoms associated with IBD.

15. The method of claim 4, further comprising monitoring the individual for changes in biomarkers associated with IBD and/or chronic pancreatitis.
